(19) United States
(12) Patent Application Publication
(10) Pub. No.: US 2005/0221275 A1
(43) Pub. Date: Oct. 6, 2005

(54) Hepatocyte free uptake assays

(76) Inventors: C. Frank Bennett, Carlsbad, CA (US); Robert McKay, Poway, CA (US); Brett P. Monia, Encinitas, CA (US); Brenda F. Baker, Carlsbad, CA (US); Namir Sioufi, Carlsbad, CA (US)

Correspondence Address: COZEN O'CONNOR, P.C., 1900 MARKET STREET, PHILADELPHIA, PA, 19103-3508, US

(21) Appl. No.: 10/824,690

(22) Filed: Apr. 14, 2004

Related U.S. Application Data
(63) Continuation-in-part of application No. 10/763,367, filed on Jan. 23, 2004.

(51) Int. Cl. C12Q 1/00; C12Q 1/68; C12N 5/06

(52) U.S. Cl. 435/4; 435/6; 435/354

(57) ABSTRACT

The present invention provides methods of identifying oligomeric compounds, such as siRNA and double-stranded RNA compounds, having bioactivity in vivo, and kits.

### CROSS-REFERENCE TO RELATED APPLICATIONS

This application is a continuation-in-part of U.S. Ser. No. 10/763,367 filed Jan. 23, 2004, which is incorporated herein by reference in its entirety.

### FIELD OF THE INVENTION

The present invention is directed, in part, to methods of identifying oligomeric compounds, such as double-stranded RNA molecules, having bioactivity in vivo and to kits therefore.

### BACKGROUND OF THE INVENTION

The concept of using antisense oligonucleotides (ASOs) to reduce protein expression was first proposed by Zamecnik and Stephenson in 1978 when they demonstrated that an oligonucleotide complementary to 13 nucleotides of the Rous sarcoma virus 35S RNA inhibited virus production in Rous infected chick embryo fibroblasts (Zamecnik et al., Proc. Natl. Acad. Sci., 1978, 75, 280-284). Advances in antisense therapeutics since this time have been substantial, with the first therapeutic ASO being approved for human use in 1998 (Marwick, J. Am. Med. Assoc., 1998, 280, 871). The recent introduction of RNA interference as a method to analyze gene function in invertibrates and plants (Fraser et al., Nature, 2000, 408, 325-330) has suggested that double-stranded RNA, specifically small nucleotide interfering RNAs (siRNAs), may also have therapeutic applications (Vickers et al., J. Biol. Chem., 2003, 278, 7108-7118).

When double-stranded RNA molecules are introduced into cells they are metabolized to small 21-23 nucleotide siRNAs with two-nucleotide (2-nt) 3′-overhangs via the endogenous ribonuclease Dicer (Grishok et al., 2000, Science, 287, 2494-2497; and Zamore et al., 2000, Cell, 101, 25-33). Inside cells, siRNA molecules bind to an RNA-induced silencing protein complex. This complex, which possesses helicase activity, unwinds the double-stranded siRNA, thereby allowing the antisense strand to bind to the targeted RNA. An endonuclease then hydrolyzes the target RNA (Zamore et al., 2000, Cell, 101, 25-33; and Zamore, 2002, Science, 296, 1265-1269). Since ultimately a single stranded RNA molecule binds to the target RNA molecule according to Watson-Crick base pairing rules, siRNA driven RNA interference is essentially an antisense mechanism of action (Vickers et al., J. Biol. Chem., 2003, 278, 7108-7118). siRNA duplexes used for silencing mammalian genes in cultured cells are usually chemically synthesized 21-23 nucleotide (21-23-nt) siRNAs, where the siRNA's sense and antisense strands are paired, containing 2-nt 3′-overhangs (Harborth et al., J. Cell. Sci., 2001, 114, 4557-4565). siRNA molecules were designed with a 2 nucleotide (2 nt) 3′-overhang because this form of siRNA has been shown to be most effective in vitro (Elbashir et al., Nature, 2001, 411, 494-498). The 5′-hydroxyl is not blocked by methylation or a 5′-phosphodiester linkage, as both prevent the 5′-phosphorylation of the antisense siRNA, a step necessary for target RNA degradation inside cells (Nykanen et al., Cell, 2001, 107, 309-321; and Schwartz et al., Mol. Cell., 2002, 10, 537-548).

Zamore and others have reported that single-stranded antisense oligonucleotides are less potent and less effective than siRNAs at reducing gene transcript levels (Zamore et al., 2000, Cell, 101, 25-33; and Caplen et al., Proc. Natl. Acad. Sci. USA, 2001, 98, 9742-9747). As the antisense molecules used in those studies were single-stranded unmodified RNA, which are rapidly degraded by endogenous nucleases, here we compare antisense siRNA molecules to ‘second generation’ phosphorothioate (PS) oligodeoxynucleotides modified to contain 2′-O-methoxyethyls (MOEs), both in vitro and in vivo. These second generation antisense oligonucleotides are chimeric molecules, which by design, contain a stretch of RNAse H sensitive 2′deoxy residues in the middle, flanked on both sides with a region of 2′MOE modifications. These molecules, termed MOE gapmers, take advantage of: 1) 2′-MOE modifications, which form higher affinity complexes and possess higher nuclease resistance relative to ‘first generation’ PS oligonucleotides, resulting in increased ASO potency both in vitro and in vivo (Altmann et al., Biochem. Soc. Trans., 1996, 24, 630-637; Dean, N. M., Pharmacology of 2′-O-(2-methoxy)-ethyl modified antisense oligonucleotides, in Antisense Technology: Principles, Strategies and Applications, S. Crooke, Editor, Marcel Dekker, 2001; and Kurreck, Eur. J. Biochem., 2003, 270, 1628-1644); and 2) PS 2′deoxyoligonucleotides, which when duplexed with RNA, serve as efficient substrates for the robust endogenous RNAse H antisense-mediated cleavage of RNA (Baker et al., Biochim. Biophys. Acta, 1999, 1489, 3-18). Indeed, antisense MOE gapmer reduction of target mRNA levels can be in the order of 85-90% of control levels (Crooke et al., Annu. Rev. Pharmacol. Toxicol., 1996, 36, 107-129; and Baker et al., Biochim. Biophys. Acta, 1999, 1489, 3-18).

Antisense oligonucleotides are known to preferentially accumulate in hepatic tissue in vivo (Cossum et al., J. Pharmacol. Exp. Ther., 1993, 267, 1181 -1190; and Graham et al., J. Pharmacol. Exp. Therap., 1998, 286, 447-458). Nestle and colleagues have previously reported that cultured hepatocytes rapidly internalize antisense compounds in the absence of cationic lipid transfection reagents (Nestle et al., J. Invest. Dermatol., 1994, 103, 569-575). These observations are likely related to the remarkable transport rates displayed by hepatocytes, where fluid-phase endocytosis at the basolateral membrane is estimated to be 8% of the total membrane surface area per minute per cell (Crawford, Semin. Liver Dis., 1996, 16, 169-189).

The present invention was undertaken to provide a primary hepatocyte cell model that would demonstrate in vitro antisense oligonucleotide uptake and intracellular trafficking similar to postulated in vivo antisense oligonucleotide uptake and trafficking. In particular, the present invention demonstrates antisense oligonucleotide mediated target mRNA reduction in primary hepatocytes without cationic lipid carriers, analogous to that postulated to occur in vivo. The results described herein suggest that the mechanism of cellular uptake of single strand MOE gapmers and double strand siRNA are different. Single strand MOE gapmers, but likely not double strand siRNA, are taken up in hepatocytes in vivo and in vitro without aid of cationic lipids. When siRNA molecules are transfected into cells, they produce a dose dependent reduction of target gene expression.

### SUMMARY OF THE INVENTION

The present invention provides methods of identifying oligomeric compounds having bioactivity in vivo. A bioindicative cell is contacted with one or more pairs of candidate oligomeric compounds in vitro. The bioindicative cell is contacted with a first oligomeric compound having a sense strand orientation. The bioindicative cell is contacted with a second oligomeric compound having an antisense strand orientation. The bioindicative cell is contacted with the second oligomeric compound at least one hour after the bioindicative cell is contacted with the first oligomeric compound. At least a portion of the second oligomeric compound is capable of hybridizing with at least a portion of the first oligomeric compound. It is determined whether the bioindicative cell has an altered phenotype. If the bioindicative cell has an altered phenotype, one or more of the pairs of candidate oligomeric compounds comprises in vivo bioactivity.

In some embodiments, the first and second oligomeric compounds are small interfering RNA. In some embodiments, the contacting occurs in the absence of a transfection reagent. In some embodiments, the bioindicative cell is a mammalian tissue-derived cell, such as a primary hepatocyte, primary keratinocyte, primary macrophage, primary fibroblast, primary pancreatic cell, or a stem cell. In some embodiments, the mammalian tissue-derived cell is a rodent or primary primate hepatocyte such as a Cynomolgus monkey or human.

In some embodiments, the altered phenotype is an increase in uptake of the candidate oligomeric compounds, decrease in expression of the mRNA produced from the gene to which the candidate oligomeric compounds are targeted, or decrease in expression of the protein encoded by the gene or mRNA to which the candidate oligomeric compounds are targeted.

In some embodiments, the bioindicative cell is contacted with the second oligomeric compound at least two hours after the bioindicative cell is contacted with the first oligomeric compound. In some embodiments, the bioindicative cell is contacted with the second oligomeric compound between two hours and four hours after the bioindicative cell is contacted with the first oligomeric compound. In some embodiments, each of the first and second oligomeric compounds comprises 10 to 40 nucleotides, 18 to 30 nucleotides, or 21 to 24 nucleotides.

In some embodiments, at least a portion of the second oligomeric compound is complementary to and capable of hybridizing to a selected target nucleic acid, the second oligomeric compound comprises a plurality of linked nucleosides linked by internucleoside linking groups, the first oligomeric compound comprises a plurality of linked nucleosides linked by internucleoside linking groups and wherein essentially each of the nucleosides is other than 2′-OH and have 3′-endo conformational geometry, and the first and second oligomeric compounds optionally comprise a phosphate group, a 3′-overhang, or a conjugate group.

The present invention also provides kits comprising an assay platform, a bioindicative cell, and one or more bioactive pairs of oligomeric compounds which comprise a first oligomeric compound having a sense strand orientation and a second oligomeric compound having an antisense strand orientation, wherein at least a portion of the second oligomeric compound is capable of hybridizing with at least a portion of the first oligomeric compound.

### BRIEF DESCRIPTION OF THE DRAWINGS

FIG. 1 shows the results of a dose-response for PTEN siRNA oligomeric compounds in primary hepatocytes.

### DESCRIPTION OF EMBODIMENTS

The present invention provides methods of identifying oligomeric compounds having bioactivity in vivo, and kits. In particular, the present invention provides methods of identifying oligomeric compounds, such as double-stranded RNA, having bioactivity in vivo. A bioindicative cell is contacted with one or more pairs of candidate oligomeric compounds in vitro. Contacting can occur by any means known to those skilled in the art. The bioindicative cell is examined to determine whether it has an altered phenotype. Such examination can be carried out via morphological analysis, biochemical analysis, or the like. If the bioindicative cell has an altered phenotype, one or more of the pairs of candidate oligomeric compounds comprises in vivo bioactivity.

In some embodiments, the oligomeric compound can be double stranded. In some embodiments, the oligomeric compound is a small interfering RNA. In some embodiments, the bioindicative cell is a mammalian tissue-derived cell including, but not limited to, a primary hepatocyte, primary keratinocyte, primary macrophage, primary fibroblast, primary pancreatic cell, or a stem cell. In some embodiments, the mammalian tissue-derived cell is a rodent (i.e., mouse or rat) primary hepatocyte. In other embodiments, the mammalian tissue-derived cell is a primate primary hepatocyte. Primates include, but are not limited to, monkeys (i.e., Cynomolgus) and humans.

Altered phenotypes include, but are not limited to, an increase in uptake of the candidate oligomeric compound, decrease in expression of the mRNA produced from the gene to which the candidate oligomeric compound is targeted, or decrease in expression of the protein encoded by the gene or mRNA to which the candidate oligomeric compound is targeted.

As used herein, “bioactivity in vivo” is any activity within a cell in vivo including, but not limited to, alteration of the level of an RNA molecule to which the oligomeric compound(s) is targeted, or alteration of a protein encoded by an RNA molecule to which the oligomeric compound(s) is targeted.

As used herein, “a bioindicative cell” is any cell in which an in vitro activity of an oligomeric compound(s) is observed and is correlated to an in vivo activity of the same oligomeric compound(s). Bioindicative cells include, but are not limited to, mammalian tissue-derived cells such as, for example, primary hepatocytes, primary keratinocytes, primary macrophages, primary fibroblasts, primary pancreatic cells, or stem cells.

As used herein, “altered phenotype” is any phenotypic trait for which an alteration can be observed. Altered phenotypes include, but are not limited to, an increase in uptake of the candidate oligomeric compound(s), decrease in expression of the RNA produced from the gene to which the candidate oligomeric compound(s) is targeted, or decrease in expression of the protein encoded by the gene to which the candidate oligomeric compound(s) is targeted.

As used herein, “transfection reagent” is any reagent that enhances transfection of an oligomeric compound(s) into a cell. Transfection reagents are well known to the skilled artisan.

As used herein, “assay platform” is any platform in which a cell-based assay can be carried out including, but not limited to, a 96-well microtiter plate, a 48-well microtiter plate, a 6-well microtiter plate, and the like.

In particular, the present invention provides methods of sequentially delivering a first oligomeric compound, such as an siRNA, comprising a sense strand orientation followed by delivery of a second oligomeric compound, such as another siRNA, comprising an antisense orientation. In some embodiments, the second oligomeric compound is delivered to the bioindicative cell at least one hour, at least two hours, or between two and four hours after delivery of the first oligomeric compound.

In some embodiments, at least the nucleic acid target region of the second oligomeric compound has 3′-endo sugar conformational geometry and comprises uniform ribofuranose nucleosides. Another suitable modification of the second oligomeric compound is a 5′-phosphate group. In some embodiments, at least one of the monomeric subunits of the hybridizing region of the first oligomeric compound are modified to give each monomeric subunit 3′-endo sugar conformational geometry. Another modification of the first oligomeric compound is a 5′-phosphate group. In some embodiments, the compositions have a double stranded region that at least in part hybridizes to and is complementary to a nucleic acid target.

In one aspect of the present invention, the second oligomeric compound is a full phosphodiester or phosphorothioate RNA that can include a 5′-phosphate group and the first oligomeric compound is a fully modified phosphodiester or phosphorothioate such that each monomeric subunit has 3′-endo sugar conformational geometry. Suitable 3′-endo modifications include, without limitation, —F, —O—CH₂CH₂—O—CH₃, —O—CH₃, —O—CH₂—CH═CH₂ or —O—CH₂—CH—CH₂—NH(Rⱼ) where Rⱼ is H or C₁-C₁₀ alkyl with 2′-O-methy as a more suitable group. The presense of modifications in both the sense and the antisense strand of compositions of the present invention greatly enhance the stability of the corresponding compositions.

In the context of this invention, the term “oligomeric compound” refers to a plurality of naturally-occurring and/or non-naturally-occurring monomeric units joined together in a specific sequence. This term includes oligonucleotides, oligonucleosides, oligonucleotide analogs, oligonucleotide mimetics, and combinations of these. Oligomeric compounds are typically structurally distinguishable from, yet functionally inter-change-able with, naturally-occurring or synthetic wild-type oligonucleotides. Thus, oligomeric compounds include all such structures that function effectively to mimic the structure and/or function of a desired RNA or DNA strand, for example, by hybridizing to a target.

Oligomeric compounds are routinely prepared linearly but can be joined or otherwise prepared to be circular and may also include branching. Oligomeric compounds can included double stranded constructs such as for example two strands hybridized to form double stranded compounds. The double stranded compounds can be linked or separate and can include overhangs on the ends. In general an oligomeric compound comprises a backbone of linked monomeric subunits where each linked monomeric subunit is directly or indirectly attached to a heterocyclic base moiety. Oligomeric compounds may also include monomeric subunits that are not linked to a heterocyclic base moiety thereby providing abasic sites. The linkages joining the monomeric subunits, the sugar moieties or surrogates and the heterocyclic base moieties can be independently modified giving rise to a plurality of motifs for the resulting oligomeric compounds including hemimers, gapmers and chimeras.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions that function similarly. Such modified or substituted oligonucleotides are often suitable over native forms because of desirable properties such as, for example, enhanced cellular uptake, enhanced affinity for nucleic acid target, and increased stability in the presence of nucleases.

Included in suitable oligomeric compounds are oligonucleotides such as antisense oligonucleotides, ribozymes, external guide sequence (EGS) oligonucleotides, alternate splicers, primers, probes, and other oligonucleotides that hybridize to at least a portion of the target nucleic acid. As such, these oligonucleotides may be introduced in the form of single-stranded, double-stranded, circular or hairpin oligonucleotides and may contain structural elements such as internal or terminal bulges or loops. Once introduced to a system, the compositions of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. Single-stranded antisense oligonucleotides that are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While a suitable form of antisense oligonucleotide is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

In the context of this invention, the term “oligonucleoside” refers to a sequence of nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S, and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference in its entirety.

In addition to the modifications described above, the nucleosides of the compositions of the invention can have a variety of other modifications so long as these other modifications either alone or in combination with other nucleosides enhance one or more of the desired properties described above. Thus, for nucleotides that are incorporated into compositions of the invention, these nucleotides can have sugar portions that correspond to naturally-occurring sugars or modified sugars. Representative modified sugars include carbocyclic or acyclic sugars, sugars having substituent groups at one or more of their 2′, 3′ or 4′ positions and sugars having substituents in place of one or more hydrogen atoms of the sugar. Additional nucleosides amenable to the present invention having altered base moieties and or altered sugar moieties are disclosed in U.S. Pat. No. 3,687,808 and PCT application PCT/US89/02323.

Oligomeric compounds having altered base moieties or altered sugar moieties are also included in the present invention. All such modified oligomeric compounds are comprehended by this invention so long as they function effectively to mimic the structure of a desired RNA or DNA strand. A class of representative base modifications include tricyclic cytosine analog, termed “G clamp” (Lin et al., J. Am. Chem. Soc., 1998, 120, 8531). This analog makes four hydrogen bonds to a complementary guanine (G) within a helix by simultaneously recognizing the Watson-Crick and Hoogsteen faces of the targeted G. This G clamp modification when incorporated into phosphorothioate oligonucleotides, dramatically enhances antisense potencies in cell culture. The compositions of the invention also can include phenoxazine-substituted bases of the type disclosed by Flanagan et al., Nat. Biotechnol., 1999, 17, 48-52.

The oligomeric compounds in accordance with this invention can comprise from about 8 to about 80 monomeric subunits (i.e. from about 8 to about 80 linked nucleosides). One of ordinary skill in the art will appreciate that the invention embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 monomeric subunits in length.

In some embodiments, the oligomeric compounds of the invention are 12 to 50 monomeric subunits in length. One having ordinary skill in the art will appreciate that this embodies oligomeric compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 monomeric subunits in length.

In other embodiments, the oligomeric compounds of the invention are 15 to 30 monomeric subunits in length. One having ordinary skill in the art will appreciate that this embodies oligomeric compounds of 15, 16, 17, 18, 19, 20, 21, 22, 23, 24, 25, 26, 27, 28, 29, or 30 monomeric subunits in length.

Oligomeric compounds used in the compositions of the present invention can also be modified to have one or more stabilizing groups that are generally attached to one or both termini of oligomeric compounds to enhance properties such as for example nuclease stability. Included in stabilizing groups are cap structures. By “cap structure” or “terminal cap moiety” is meant chemical modifications, which have been incorporated at either terminus of oligonucleotides (see for example Wincott et al., WO 97/26270, incorporated by reference herein). These terminal modifications protect the oligomeric compounds having terminal nucleic acid molecules from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl)nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

Particularly suitable 3′-cap structures of the present invention include, for example, 4′,5′-methylene nucleotide; 1-(beta-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925; incorporated by reference herein).

A further embodiment of the present invention is “structured” antisense constructs, e.g. siRNA, which contain suitable and/or enabling attributes and compositions for regulation of gene expression in vivo through usage of conventional administration procedures. One primary feature of the structured constructs is that they exist in both a structured and unstructured form under physiological conditions, e.g. unhybridized single-strand and hybridized double-strand forms. The antisense construct may be modified to impart resistance to degradation by nucleases in either or both forms.

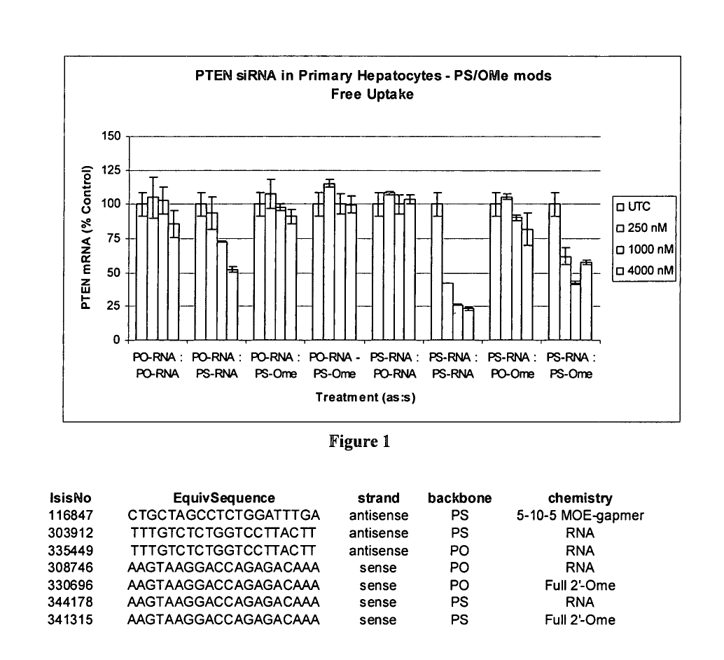

Modifications to the base, sugar, and phosphate linkage may also be used to affect changes in the equilibrium between the structured and unstructured form, in particular for sequences or compositions that yield duplex stabilities below or above the optimal range for in vivo delivery applications, e.g. Tm=37±8° C. These modifications may include bases that form mismatches, with preference for mismatched bases in the sense portion of the antisense construct. See FIG. 1.

It is not necessary for all positions in an oligomeric compound to be uniformly modified, and in fact more than one of the aforementioned modifications may be incorporated in a single oligomeric compound or even at a single monomeric subunit such as a nucleoside within an oligonucleotide. The present invention also includes chimeric oligomeric compounds such as chimeric oligonucleotides. “Chimeric” oligomeric compounds or “chimeras,” in the context of this invention, are oligomeric compounds such as oligonucleotides containing two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligonucleotides when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric compositions of the invention may be formed as composite structures of two or more oligomeric compounds such as oligonucleotides, oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

Specific examples of suitable oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

In the C. elegans system, modification of the internucleoside linkage (phosphorothioate) did not significantly interfere with RNAi activity. Based on this observation, it is suggested that certain compositions of the invention can also have one or more modified internucleoside linkages. A suitable phosphorus containing modified internucleoside linkage is the phosphorothioate internucleoside linkage.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleoside linkages is a 3′ to 3′, 5′ to 5′ or 2′ to 2′ linkage. Suitable oligonucleotides having inverted polarity comprise a single 3′ to 3′ linkage at the 3′-most internucleoside linkage, i.e. a single inverted nucleoside residue that may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In some embodiments of the invention, oligonucleotides have one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleoside linkage is represented as —O—P(═O)(OH)—O—CH₂—). The MMI type internucleoside linkages are disclosed in the above referenced U.S. Pat. No. 5,489,677. Suitable amide internucleoside linkages are disclosed in the above referenced U.S. Pat. No. 5,602,240.

Suitable modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference in its entirety.

In addition to having a 2′-O-methyl modified nucleiside the compositions of the present invention may also contain additional modified sugar moieties. Suitable modified sugar moieties comprise a sugar substituent group selected from: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH₂)ₙO)ₘ—CH₃, O(CH₂)ₙOCH₃, O(CH₂)ₙNH₂, O(CH₂)ₙCH₃, O(CH₂)ₙONH₂, and O(CH₂)ₙON—((CH₂)ₙCH₃)₂, where n and m are from 1 t about 10. Other suitable sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocyclo-alkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy(2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification includes 2′-dimethylamino-oxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy(—O—CH₃), aminopropoxy(—OCH₂CH₂CH₂NH₂), allyl(-CH₂—CH═CH₂), —O-allyl(-O—CH₂—CH═CH₂) and fluoro (F). 2′-Sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Particularly suitable sugar substituent groups include O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃))₂, where n and m are from 1 to about 10.

Further representative sugar substituent groups include groups of formula Iₐ or IIₐ: 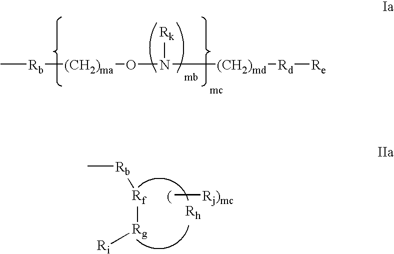
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ; 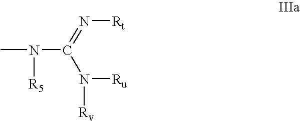
Rₚ and R_{q} are each independently hydrogen or C₁-C₁₀ alkyl;
Rᵣ is —Rₓ—R_{y};
each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₚ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where the acyl is an acid amide or an ester;
or Rₘ and Rₙ, together, are a nitrogen protecting group, are joined in a ring structure that optionally includes an additional heteroatom selected from N and O or are a chemical functional group;
Rⱼ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;
mₐ is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Oligomeric compounds that are Conformationally Preorganized,” hereby incorporated by reference in its entirety.

Representative guanidino substituent groups that are shown in formula III and IV are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 that is hereby incorporated by reference in its entirety.

Representative dimethylaminoethyloxyethyl substituent groups are disclosed in International Patent Application PCT/US99/17895, entitled “2′-O-Dimethylaminoethyloxy-ethyl-Oligomeric compounds”, filed Aug. 6, 1999, hereby incorporated by reference in its entirety.

Oligomeric compounds including oligonucleotides may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C≡—C—CH₃)uracil and cytosine and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyl-adenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the compositions of the invention. These include 5-substituted pyrimidines, 6-azapyrimidines and N-2, N-6 and O-6 substituted purines, including 2 aminopropyladenine, 5-propynyluracil and 5-propynylcytosine. 5 methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently suitable base substitutions, even more particularly when combined with 2′-O-methoxyethyl sugar modifications.

Oligomeric compounds of the present invention can also include polycyclic heterocyclic compounds in place of one or more heterocyclic base moieties. A number of tricyclic heterocyclic comounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

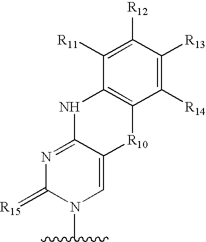

Representative cytosine analogs that make 3 hydrogen bonds with a guanosine in a second strand include 1,3-diazaphenoxazine-2-one (R₁₀═O, R₁₁—R₁₄═H) (Kurchavov, et al., Nucleosides and Nucleotides, 1997, 16, 1837-1846), 1,3-diazaphenothiazine-2-one (R₁₀═S, R₁₁—R₁₄═H), (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (R₁₀═O, R₁₁—R₁₄═F) (Wang, J.; Lin, K.-Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. patent application entitled “Modified Peptide Nucleic Acids” filed May 24, 2002, Ser. No. 10/155,920; and U.S. patent application entitled “Nuclease Resistant Chimeric Oligonucleotides” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀₌O, R₁₁=—O—(CH₂)₂—NH₂, R₁₂₋₁₄═H ) (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. Ser. No. 6,028,183, which issued on May 22, 2000, and U.S. Pat. Ser. No. 6,007,992, which issued on Dec. 28, 1999, the contents of both are commonly assigned with this application and are incorporated herein in their entirety.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity (Lin, K-Y; Matteucci, M. J. Am. Chem. Soc. 1998, 120, 8531-8532). The activity enhancement was even more pronounced in case of G-clamp, as a single substitution was shown to significantly improve the in vitro potency of a 20 mer 2′-deoxyphosphorothioate oligonucleotides (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518). Nevertheless, to optimize oligonucleotide design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. patent application Ser. No. 09/996,292 filed Nov. 28, 2001, certain of which are commonly owned with the instant application, and each of which is herein incorporated by reference.

Oligomeric compounds used in the compositions of the present invention can also be modified to have one or more moieties or conjugates which enhance the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmaco-dynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

The oligomeric compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

In one aspect of the present invention oligomeric compounds include nucleosides synthetically modified to induce a 3′-endo sugar conformation. A nucleoside can incorporate synthetic modifications of the heterocyclic base moiety, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement of RNA interference which is supported in part by the fact that duplexes composed of 2′-deoxy-2′-F-nucleosides appear efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric compounds having one or more nucleosides modified in such a way as to favor a C3′-endo type conformation.

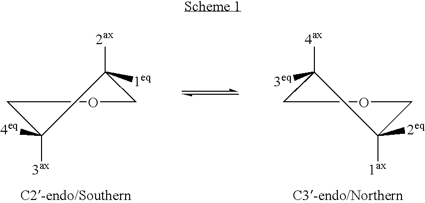

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element, as illustrated in FIG. 2, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Some modifications actually lock the conformational geometry by formation of a bicyclic sugar moiety e.g. locked nucleic acid (LNA, Singh et al, Chem. Commun. (1998), 4, 455-456), and ethylene bridged nucleic acids (ENA, Morita et al, Bioorganic & Medicinal Chemistry Letters (2002), 12, 73-76.)

Examples of modified nucleosides amenable to the present invention are shown below in Table 1. These examples are meant to be representative and not exhaustive.

TABLE 1

| | | |
|---|---|---|
| 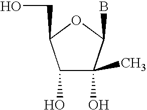 | 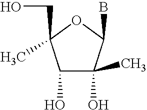 | 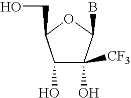 |
| 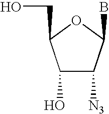 | 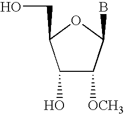 | 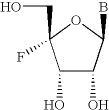 |
| 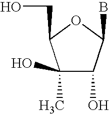 | 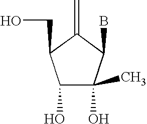 | 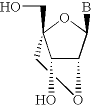 |
| 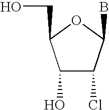 | 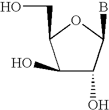 | 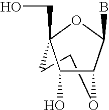 |
| 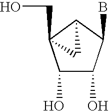 | 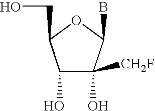 | 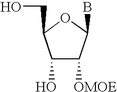 |
| 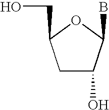 | 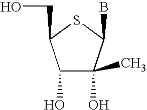 | 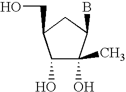 |
| 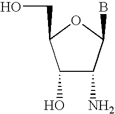 | | |

Suitable conformations of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in one or more of the oligomeric compounds of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below.) Nucleosides known to be inhibitors/substrates for RNA dependent RNA polymerases (for example HCV NS5B).

In one aspect, the present invention is directed to oligomeric compounds that are prepared having enhanced properties compared to native RNA against nucleic acid targets. A target is identified and an oligomeric compound is selected having an effective length and sequence that is complementary to a portion of the target sequence. Each nucleoside of the selected sequence is scrutinized for possible enhancing modifications. A suitable modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligomeric compound. The selected sequence can be further divided into regions and the nucleosides of each region evaluated for enhancing modifications that can be the result of a chimeric configuration. Consideration is also given to the termini (e.g. 5′ and 3′-termini) as there are often advantageous modifications that can be made to one or more of the terminal monomeric subunits. In one aspect of the invention, desired properties and or activity of oligomeric compounds are enhanced by the inclusion of a 5′-phosphate or modified phosphate moiety.

The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and O4′-endo pucker. This is consistent with Berger, et. al., Nucleic Acids Research, 1998, 26, 2473-2480, who pointed out that in considering the furanose conformations which give rise to B-form duplexes consideration should also be given to a O4′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic strand of oligomeric compound to an RNA target strand. In the case of antisense, effective inhibition of the mRNA requires that the antisense DNA have a very high binding affinity with the mRNA. Otherwise the desired interaction between the synthetic strand and target mRNA strand will occur infrequently, resulting in decreased efficacy.

One routinely used method of modifying the sugar puckering is the substitution of the sugar at the 2′-position with a substituent group that influences the sugar geometry. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (2′-deoxy-2′-fluoroadenosine-2′-deoxy-2′-fluoro-adenosine) is further correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligonucleotides having the 2′-MOE modification displayed improved RNA affinity and higher nuclease resistance. Chimeric oligonucleotides having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states. One such MOE substituted oligonucleotide is presently being investigated in clinical trials for the treatment of CMV retinitis.

To better understand the higher RNA affinity of 2′-O-methoxyethyl substituted RNA and to examine the conformational properties of the 2′-O-methoxyethyl substituent, two dodecamer oligonucleotides were synthesized having SEQ ID NO: 46 (CGC GAA UUC GCG) and SEQ ID NO: 47 (GCG CUU AAG CGC). These self-complementary strands have every 2′-position modified with a 2′-O-methoxyethyl. The duplex was crystallized at a resolution of 1.7 Ångstrom and the crystal structure was determined. The conditions used for the crystallization were 2 mM oligonucleotide, 50 mM Na Hepes pH 6.2-7.5, 10.50 mM MgCl₂, 15% PEG 400. The crystal data showed: space group C2, cell constants a=41.2 Å, b=34.4 Å, c=46.6 Å,. =92.4°. The resolution was 1.7 Å at −170° C. The current R=factor was 20% (R′free 26%).

This crystal structure is believed to be the first crystal structure of a fully modified RNA oligonucleotide analogue. The duplex adopts an overall A-form conformation and all modified sugars display C3′-endo pucker. In most of the 2′-O-substituents, the torsion angle around the A′-B′ bond, as depicted in Structure II below, of the ethylene glycol linker has a gauche conformation. For 2′-O-MOE, A′ and B′ of Structure II below are methylene moieties of the ethyl portion of the MOE and R′ is the methoxy portion.

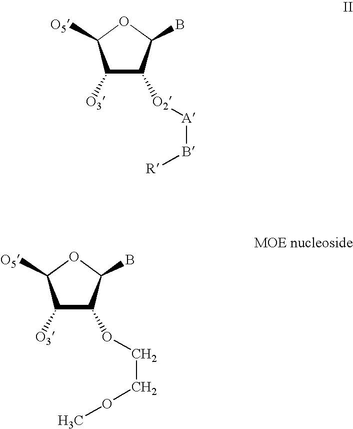

In the crystal, the 2′-O-MOE RNA duplex adopts a general orientation such that the crystallographic 2-fold rotation axis does not coincide with the molecular 2-fold rotation axis. The duplex adopts the expected A-type geometry and all of the 24 2′-O-MOE substituents were visible in the electron density maps at full resolution. The electron density maps as well as the temperature factors of substituent atoms indicate flexibility of the 2′-O-MOE substituent in some cases.

Most of the 2′-O-MOE substituents display a gauche conformation around the C—C bond of the ethyl linker. However, in two cases, a trans conformation around the C—C bond is observed. The lattice interactions in the crystal include packing of duplexes against each other via their minor grooves. Therefore, for some residues, the conformation of the 2′-O-substituent is affected by contacts to an adjacent duplex. In general, variations in the conformation of the substituents (e.g. g⁺ or g⁻ around the C—C bonds) create a range of interactions between substituents, both inter-strand, across the minor groove, and intra-strand. At one location, atoms of substituents from two residues are in van der Waals contact across the minor groove. Similarly, a close contact occurs between atoms of substituents from two adjacent intra-strand residues.

Previously determined crystal structures of A-DNA duplexes were for those that incorporated isolated 2′-O-methyl T residues. In the crystal structure noted above for the 2′-O-MOE substituents, a conserved hydration pattern has been observed for the 2′-O-MOE residues. A single water molecule is seen located between O2′, O3′ and the methoxy oxygen atom of the substituent, forming contacts to all three of between 2.9 and 3.4 Å. In addition, oxygen atoms of substituents are involved in several other hydrogen bonding contacts. For example, the methoxy oxygen atom of a particular 2′-O-substituent forms a hydrogen bond to N3 of an adenosine from the opposite strand via a bridging water molecule.

In several cases a water molecule is trapped between the oxygen atoms O2′, O3′ and OC′ of modified nucleosides. 2′-O-MOE substituents with trans conformation around the C—C bond of the ethylene glycol linker are associated with close contacts between OC′ and N2 of a guanosine from the opposite strand, and, water-mediated, between OC′ and N3(G). When combined with the available thermodynamic data for duplexes containing 2′-O-MOE modified strands, this crystal structure allows for further detailed structure-stability analysis of other modifications.

In extending the crystallographic structure studies, molecular modeling experiments were performed to study further enhanced binding affinity of oligonucleotides having 2′-O-modifications. The computer simulations were conducted on compounds of SEQ ID NO: 46, above, having 2′-O-modifications located at each of the nucleosides of the oligonucleotide. The simulations were performed with the oligonucleotide in aqueous solution using the AMBER force field method (Cornell et al., J. Am. Chem. Soc., 1995, 117, 5179-5197)(modeling software package from UCSF, San Francisco, Calif.). The calculations were performed on an Indigo2 SGI machine (Silicon Graphics, Mountain View, Calif.).

Further 2′-O-modifications that will have a 3′-endo sugar influence include those having a ring structure that incorporates a two atom portion corresponding to the A′ and B′ atoms of Structure II. The ring structure is attached at the 2′ position of a sugar moiety of one or more nucleosides that are incorporated into an oligonucleotide. The 2′-oxygen of the nucleoside links to a carbon atom corresponding to the A′ atom of Structure II. These ring structures can be aliphatic, unsaturated aliphatic, aromatic or heterocyclic. A further atom of the ring (corresponding to the B′ atom of Structure II), bears a further oxygen atom, or a sulfur or nitrogen atom. This oxygen, sulfur or nitrogen atom is bonded to one or more hydrogen atoms, alkyl moieties, or haloalkyl moieties, or is part of a further chemical moiety such as a ureido, carbamate, amide or amidine moiety. The remainder of the ring structure restricts rotation about the bond joining these two ring atoms. This assists in positioning the “further oxygen, sulfur or nitrogen atom” (part of the R position as described above) such that the further atom can be located in close proximity to the 3′-oxygen atom (O3′) of the nucleoside.

Another suitable 2′-sugar substituent group that gives a 3′-endo sugar conformational geometry is the 2′-OMe group. 2′-Substitution of guanosine, cytidine, and uridine dinucleoside phosphates with the 2′-OMe group showed enhanced stacking effects with respect to the corresponding native (2′-OH) species leading to the conclusion that the sugar is adopting a C3′-endo conformation. In this case, it is believed that the hydrophobic attractive forces of the methyl group tend to overcome the destabilizing effects of its steric bulk.

The ability of oligonucleotides to bind to their complementary target strands is compared by determining the melting temperature (Tₘ) of the hybridization complex of the oligonucleotide and its complementary strand. The melting temperature (Tₘ), a characteristic physical property of double helices, denotes the temperature (in degrees centigrade) at which 50% helical (hybridized) versus coil (unhybridized) forms are present. Tₘ is measured by using the UV spectrum to determine the formation and breakdown (melting) of the hybridization complex. Base stacking, which occurs during hybridization, is accompanied by a reduction in UV absorption (hypochromicity). Consequently, a reduction in UV absorption indicates a higher Tₘ. The higher the Tₘ, the greater the strength of the bonds between the strands.

Freier and Altmann, Nucleic Acids Research, (1997) 25:4429-4443, have previously published a study on the influence of structural modifications of oligonucleotides on the stability of their duplexes with target RNA. In this study, the authors reviewed a series of oligonucleotides containing more than 200 different modifications that had been synthesized and assessed for their hybridization affinity and Tm. Sugar modifications studied included substitutions on the 2′-position of the sugar, 3′-substitution, replacement of the 4′-oxygen, the use of bicyclic sugars, and four member ring replacements. Several nucleobase modifications were also studied including substitutions at the 5, or 6 position of thymine, modifications of pyrimidine heterocycle and modifications of the purine heterocycle. Modified internucleoside linkages were also studied including neutral, phosphorus and non-phosphorus containing internucleoside linkages.

Increasing the percentage of C3′-endo sugars in a modified oligonucleotide targeted to an RNA target strand should preorganize this strand for binding to RNA. Of the several sugar modifications that have been reported and studied in the literature, the incorporation of electronegative substituents such as 2′-fluoro or 2′-alkoxy shift the sugar conformation towards the 3′ endo (northern) pucker conformation. This preorganizes an oligonucleotide that incorporates such modifications to have an A-form conformational geometry. This A-form conformation results in increased binding affinity of the oligonucleotide to a target RNA strand.

Molecular modeling experiments were performed to study further enhanced binding affinity of oligonucleotides having 2′-O-modifications. Computer simulations were conducted on compounds having SEQ ID NO:46, r(CGC GAA UUC GCG), having 2′-O-modifications of the invention located at each of the nucleoside of the oligonucleotide. The simulations were performed with the oligonucleotide in aqueous solution using the AMBER force field method (Cornell et al., J. Am. Chem. Soc., 1995, 117, 5179-5197)(modeling software package from UCSF, San Francisco, Calif.). The calculations were performed on an Indigo2 SGI machine (Silicon Graphics, Mountain View, Calif.).

In addition, for 2′-substituents containing an ethylene glycol motif, a gauche interaction between the oxygen atoms around the O—C—C—O torsion of the side chain may have a stabilizing effect on the duplex (Freier ibid.). Such gauche interactions have been observed experimentally for a number of years (Wolfe et al., Acc. Chem. Res., 1972, 5, 102; Abe et al., J. Am. Chem. Soc., 1976, 98, 468). This gauche effect may result in a configuration of the side chain that is favorable for duplex formation. The exact nature of this stabilizing configuration has not yet been explained. While we do not want to be bound by theory, it may be that holding the O—C—C—O torsion in a single gauche configuration, rather than a more random distribution seen in an alkyl side chain, provides an entropic advantage for duplex formation.

Representative 2′-substituent groups amenable to the present invention that give A-form conformational properties (3′-endo) to the resultant duplexes include 2′-O-alkyl, 2′-O-substituted alkyl and 2′-fluoro substituent groups. Suitable substituent groups are various alkyl and aryl ethers and thioethers, amines and monoalkyl and dialkyl substituted amines. It is further intended that multiple modifications can be made to one or more of the oligomeric compounds of the invention at multiple sites of one or more monomeric subunits (nucleosides are suitable) and or internucleoside linkages to enhance properties such as but not limited to activity in a selected application. Tables 2 through 8 list nucleoside and internucleoside linkage modifications/replacements that have been shown to give a positive εTm per modification when the modification/replacement was made to a DNA strand that was hybridized to an RNA complement.

TABLE 2
Modified DNA strand having 2′-substituent groups that gave an overall
increase in Tm against an RNA complement:

| | Positive ∈Tm/mod |
|---|---|
| 2′-substituents | 2′-OH |
| | 2′-O—C₁-C₄ alkyl |
| | 2′-O—(CH₂)₂CH₃ |
| | 2′-O—CH₂CH═CH₂ |
| | 2′-F |
| | 2′-O—(CH₂)₂—O—CH₃ |
| | 2′-(O—(CH₂)₂)₂—O—CH₃ |
| | 2′-(O—(CH₂)₂)₃—O—CH₃ |
| | 2′-(O—(CH₂)₂)₄—O—CH₃ |
| | 2′-(O—(CH₂)₂)₃—O—(CH₂)₈CH₃ |
| | 2′-O—(CH₂)₂CF₃ |
| | 2′-O—(CH₂)₂OH |
| | 2′-O—(CH₂)₂F |
| | 2′-O—CH₂CH(CH₃)F |
| | 2′-O—CH₂CH(CH₂OH)OH |
| | 2′-O—CH₂CH(CH₂OCH₃)OCH₃ |
| | 2′-O—CH₂CH(CH₃)OCH₃ |
| | 2′-O—CH₂—C₁₄H₇O₂(—C₁₄H₇O₂ = Anthraquinone) |
| | 2′-O—(CH₂)₃—NH₂* |
| | 2′-O—(CH₂)₄—NH₂* |

*These modifications can increase the Tm of oligonucleotides but can also decrease the Tm depending on positioning and number (motiff dependant).

TABLE 3
Modified DNA strand having modified sugar ring (see structure x)
that gave an
overall increase in Tm against an RNA complement:

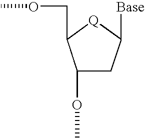

| | Positive εTm/mod |
|---|---|
| Q | —S— |
| | —CH₂— |

Note: In general ring oxygen substitution with sulfur or methylene had only a minor effect on Tm for the specific motiffs studied. Substitution at the 2′-position with groups shown to stabilize the duplex were destabilizing when CH₂ replaced the ring O. This is thought to be due to the necessary gauche interaction between the ring O with particular 2′- substituents (for example —O—CH₃ and —(O—CH₂CH₂)₃—O—CH₃.

TABLE 4
Modified DNA strand having modified sugar ring that give
an overall increase
in Tm against an RNA complement:

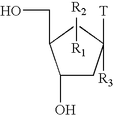

Positive εTm/mod

| | |
|---|---|
| —C(H)R₁ effects | OH |
| (R₂, R₃ both = H) | CH₃* |
| | CH₂OH* |
| | OCH₃* |

*These modifications can increase the Tm of oligonucleotides but can also decrease the Tm depending on positioning and number (motiff dependant).

TABLE 5
Modified DNA strand having bicyclic substitute
sugar modifications that give
an overall increase in Tm against an RNA complement:

| Formula | Positive εTm/mod |
|---|---|
| I | + |
| II | + |

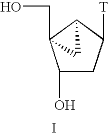

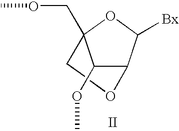

TABLE 6
Modified DNA strand having modified heterocyclic
base moieties that give an
overall increase in Tm against an RNA complement:

| Modification/Formula | Positive εTm/mod |
|---|---|
| Heterocyclic base | 2-thioT |
| modifications | 2′-O-methylpseudoU |
| | 7-halo-7-deaza purines |
| | 7-propyne-7-deaza purines |
| | 2-aminoA(2,6-diaminopurine) |
| 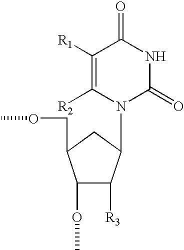 | |
| R₂, R₃ = H), R₁ = | Br |
| | C/C—CH₃ |
| | (CH₂)₃NH₂ |
| | CH₃ |
| Motiffs-disubstitution | |
| R₁ = C/C—CH₃, R₂ = H, R₃ = | F |
| R₁ = C/C—CH₃, R₂ = H | R₃ = O—(CH₂)₂—O—CH₃ |
| R₁ = O—CH₃, R₂ = H, | R₃ = O—(CH₂)₂—O—CH_{3*} |

*This modification can increase the Tm of oligonucleotides but can also decrease the Tm depending on positioning and number (motiff dependant).

Substitution at R₁ can be stabilizing, substitution at R₂ is generally greatly destabilizing (unable to form anti conformation), motiffs with stabilizing 5 and 2′-substituent groups are generally additive e.g. increase stability.

Substitution of the O4 and O2 positions of 2′-O-methyl uridine was greatly duplex destabilizing as these modifications remove hydrogen binding sites that would be an expected result. 6-Aza T also showed extreme destabilization as this substitution reduces the pKₐ and shifts the nucleoside toward the enol tautomer resulting in reduced hydrogen bonding.

TABLE 7
DNA strand having at least one modified phosphorus
containing internucleoside linkage and the effect
on the Tm against an RNA complement:

| ∈Tm/mod+ | ∈Tm/mod− |
|---|---|
| phosphoramidate (the 3′-bridging | phosphorothioate¹ |
| atom replaced with an N(H)R | phosphoramidate¹ |
| group, stabilization effect | methyl phosphonates¹ |
| enhanced when also have 2′-F) | |

(¹one of the non-bridging oxygen atoms replaced with S, N(H)R or —CH₃)

TABLE 8
DNA strand having at least one non-phosphorus containing internucleoside
linkage and the effect on the Tm against an RNA complement:
Positive ∈Tm/mod

—CH₂C(═O)NHCH₂—*
—CH₂C(═O)N(CH₃)CH₂—*
—CH₂C(═O)N(CH₂CH₂CH₃)CH₂—*
—CH₂C(═O)N(H)CH₂— (motiff with 5′-propyne on T's)
—CH₂N(H)C(═O)CH₂—*
—CH₂N(CH₃)OCH₂—*
—CH₂N(CH₃)N(CH₃)CH₂—*
*This modification can increase the Tm of oligonucleotides but can also decrease the Tm depending on positioning and number (motiff dependant).
Notes:
In general carbon chain internucleotide linkages were destabilizing to duplex formation. This destabilization was not as severe when double and tripple bonds were utilized. The use of glycol and flexible ether linkages were also destabilizing.

Suitable ring structures of the invention for inclusion as a 2′-O modification include cyclohexyl, cyclopentyl and phenyl rings as well as heterocyclic rings having spacial footprints similar to cyclohexyl, cyclopentyl and phenyl rings. Particularly suitable 2′-O-substituent groups of the invention are listed below including an abbreviation for each:
2′-O-(trans 2-methoxy cyclohexyl)—2′-O-(TMCHL)
2′-O-(trans 2-methoxy cyclopentyl)—2′-O-(TMCPL)
2′-O-(trans 2-ureido cyclohexyl)—2′-O-(TUCHL)
2′-O-(trans 2-methoxyphenyl)—2′-O-(2MP)

Structural details for duplexes incorporating such 2-O-substituents were analyzed using the described AMBER force field program on the Indigo2 SGI machine. The simulated structure maintained a stable A-form geometry throughout the duration of the simulation. The presence of the 2′ substitutions locked the sugars in the C3′-endo conformation.

The simulation for the TMCHL modification revealed that the 2′-O-(TMCHL) side chains have a direct interaction with water molecules solvating the duplex. The oxygen atoms in the 2′-O-(TMCHL) side chain are capable of forming a water-mediated interaction with the 3′ oxygen of the phosphate backbone. The presence of the two oxygen atoms in the 2′-O-(TMCHL) side chain gives rise to favorable gauche interactions. The barrier for rotation around the O—C—C—O torsion is made even larger by this novel modification. The preferential preorganization in an A-type geometry increases the binding affinity of the 2′-O-(TMCHL) to the target RNA. The locked side chain conformation in the 2′-O-(TMCHL) group created a more favorable pocket for binding water molecules. The presence of these water molecules played a key role in holding the side chains in the preferable gauche conformation. While not wishing to be bound by theory, the bulk of the substituent, the diequatorial orientation of the substituents in the cyclohexane ring, the water of hydration and the potential for trapping of metal ions in the conformation generated will additionally contribute to improved binding affinity and nuclease resistance of oligonucleotides incorporating nucleosides having this 2′-O-modification.

As described for the TMCHL modification above, identical computer simulations of the 2′-O-(TMCPL), the 2′-O-(2MP) and 2′-O-(TUCHL) modified oligonucleotides in aqueous solution also illustrate that stable A-form geometry will be maintained throughout the duration of the simulation. The presence of the 2′ substitution will lock the sugars in the C3′-endo conformation and the side chains will have direct interaction with water molecules solvating the duplex. The oxygen atoms in the respective side chains are capable of forming a water-mediated interaction with the 3′ oxygen of the phosphate backbone. The presence of the two oxygen atoms in the respective side chains give rise to the favorable gauche interactions. The barrier for rotation around the respective O—C—C—O torsions will be made even larger by respective modification. The preferential preorganization in A-type geometry will increase the binding affinity of the respective 2′-O-modified oligonucleotides to the target RNA. The locked side chain conformation in the respective modifications will create a more favorable pocket for binding water molecules. The presence of these water molecules plays a key role in holding the side chains in the preferable gauche conformation. The bulk of the substituent, the diequatorial orientation of the substituents in their respective rings, the water of hydration and the potential trapping of metal ions in the conformation generated will all contribute to improved binding affinity and nuclease resistance of oligonucleotides incorporating nucleosides having these respective 2′-O-modification.

Ribose conformations in C2′-modified nucleosides containing S-methyl groups were examined. To understand the influence of 2′-O-methyl and 2′-S-methyl groups on the conformation of nucleosides, we evaluated the relative energies of the 2′-O— and 2′-S-methylguanosine, along with normal deoxyguanosine and riboguanosine, starting from both C2′-endo and C3′-endo conformations using ab initio quantum mechanical calculations. All the structures were fully optimized at HF/6-31G* level and single point energies with electron-correlation were obtained at the MP2/6-31G*//HF/6-31G* level. As shown in Table 9, the C2′-endo conformation of deoxyguanosine is estimated to be 0.6 kcal/mol more stable than the C3′-endo conformation in the gas-phase. The conformational preference of the C2′-endo over the C3′-endo conformation appears to be less dependent upon electron correlation as revealed by the MP2/6-31G*//HF/6-31G* values which also predict the same difference in energy. The opposite trend is noted for riboguanosine. At the HF/6-31G* and MP2/6-31G*//HF/6-31G* levels, the C3′-endo form of riboguanosine is shown to be about 0.65 and 1.41 kcal/mol more stable than the C2′endo form, respectively.

TABLE 9
Relative energies* of the C3′-endo and C2′-endo
conformations of representative nucleosides.

| HF/6-31G | MP2/6-31-G | CONTINUUM | AMBER | MODEL |
|---|---|---|---|---|
| dG | 0.60 | 0.56 | 0.88 | 0.65 |
| rG | −0.65 | −1.41 | −0.28 | −2.09 |
| 2′-O-MeG | −0.89 | −1.79 | −0.36 | −0.86 |
| 2′-S-MeG | 2.55 | 1.41 | 3.16 | 2.43 |

*energies are in kcal/mol relative to the C2′-endo conformation

Table 9 also includes the relative energies of 2′-O-methylguanosine and 2′-S-methylguanosine in C2′-endo and C3′-endo conformation. This data indicates the electronic nature of C2′-substitution has a significant impact on the relative stability of these conformations. Substitution of the 2′-O-methyl group increases the preference for the C3′-endo conformation (when compared to riboguanosine) by about 0.4 kcal/mol at both the HF/6-31G* and MP2/6-31G*//HF/6-31G* levels. In contrast, the 2′-S-methyl group reverses the trend. The C2′-endo conformation is favored by about 2.6 kcal/mol at the HF/6-31G* level, while the same difference is reduced to 1.41 kcal/mol at the MP2/6-31G*//HF/6-31G* level. For comparison, and also to evaluate the accuracy of the molecular mechanical force-field parameters used for the 2′-O-methyl and 2′-S-methyl substituted nucleosides, we have calculated the gas phase energies of the nucleosides. The results reported in Table 9 indicate that the calculated relative energies of these nucleosides compare qualitatively well with the ab initio calculations.

Additional calculations were also performed to gauge the effect of solvation on the relative stability of nucleoside conformations. The estimated solvation effect using HF/6-31G* geometries confirms that the relative energetic preference of the four nucleosides in the gas-phase is maintained in the aqueous phase as well (Table 9). Solvation effects were also examined using molecular dynamics simulations of the nucleosides in explicit water. From these trajectories, one can observe the predominance of C2′-endo conformation for deoxyriboguanosine and 2′-S-methylriboguanosine while riboguanosine and 2′-O-methylriboguanosine prefer the C3′-endo conformation. These results are in much accord with the available NMR results on 2′-S-methylribonucleosides. NMR studies of sugar puckering equilibrium using vicinal spin-coupling constants have indicated that the conformation of the sugar ring in 2′-S-methylpyrimidine nucleosides show an average of >75% S-character, whereas the corresponding purine analogs exhibit an average of >90% S-pucker (Fraser, A., Wheeler, P., Cook, P. D. and Sanghvi, Y. S., J. Heterocycl. Chem., 1993, 30, 1277-1287). It was observed that the 2′-S-methyl substitution in deoxynucleoside confers more conformational rigidity to the sugar conformation when compared with deoxyribonucleosides.

Structural features of DNA:RNA, OMe-DNA:RNA and SMe-DNA:RNA hybrids were also observed. The average RMS deviation of the DNA:RNA structure from the starting hybrid coordinates indicate the structure is stabilized over the length of the simulation with an approximate average RMS deviation of 1.0 Å. This deviation is due, in part, to inherent differences in averaged structures (i.e. the starting conformation) and structures at thermal equilibrium. The changes in sugar pucker conformation for three of the central base pairs of this hybrid are in good agreement with the observations made in previous NMR studies. The sugars in the RNA strand maintain very stable geometries in the C3′-endo conformation with ring pucker values near 0°. In contrast, the sugars of the DNA strand show significant variability.

The average RMS deviation of the OMe-DNA:RNA is approximately 1.2 Å from the starting A-form conformation; while the SMe-DNA:RNA shows a slightly higher deviation (approximately 1.8 Å) from the starting hybrid conformation. The SMe-DNA strand also shows a greater variance in RMS deviation, suggesting the S-methyl group may induce some structural fluctuations. The sugar puckers of the RNA complements maintain C3′-endo puckering throughout the simulation. As expected from the nucleoside calculations, however, significant differences are noted in the puckering of the OMe-DNA and SMe-DNA strands, with the former adopting C3′-endo, and the latter, C1′-exo/C2′-endo conformations.

An analysis of the helicoidal parameters for all three hybrid structures has also been performed to further characterize the duplex conformation. Three of the more important axis-basepair parameters that distinguish the different forms of the duplexes, X-displacement, propeller twist, and inclination, are reported in Table 10. Usually, an X-displacement near zero represents a B-form duplex; while a negative displacement, which is a direct measure of deviation of the helix from the helical axis, makes the structure appear more A-like in conformation. In A-form duplexes, these values typically vary from −4 Å to −5 Å. In comparing these values for all three hybrids, the SMe_DNA:RNA hybrid shows the most deviation from the A-form value, the OMe_DNA:RNA shows the least, and the DNA:RNA is intermediate. A similar trend is also evident when comparing the inclination and propeller twist values with ideal A-form parameters. These results are further supported by an analysis of the backbone and glycosidic torsion angles of the hybrid structures. Glycosidic angles (X) of A-form geometries, for example, are typically near −159° while B form values are near −102°. These angles are found to be −162°, −133°, and −108° for the OMe-DNA, DNA, and SMe-DNA strands, respectively. All RNA complements adopt an X angle close to −160°. In addition, “crankshaft” transitions were also noted in the backbone torsions of the central UpU steps of the RNA strand in the SMe-DNA:RNA and DNA;RNA hybrids. Such transitions suggest some local conformational changes may occur to relieve a less favorable global conformation. Taken overall, the results indicate the amount of A-character decreases as OMe-DNA:RNA>DNA:RNA>SMe-DNA:RNA, with the latter two adopting more intermediate conformations when compared to A- and B-form geometries.

TABLE 10
Average helical parameters derived from the last 500 ps of simulation time.
(canonical A-and B-form values are given for comparison)

| Helicoidal | B-DNA | B-DNA | A-DNA | | | |
|---|---|---|---|---|---|---|
| Parameter | (x-ray) | (fibre) | (fibre) | DNA:RNA | OMe_DNA:RNA | SMe_DNA:RNA |
| X-disp | 1.2 | 0.0 | −5.3 | −4.5 | −5.4 | −3.5 |
| Inclination | −2.3 | 1.5 | 20.7 | 11.6 | 15.1 | 0.7 |
| Propeller | −16.4 | −13.3 | −7.5 | −12.7 | −15.8 | −10.3 |

Stability of C2′-modified DNA:RNA hybrids was determined. Although the overall stability of the DNA:RNA hybrids depends on several factors including sequence-dependencies and the purine content in the DNA or RNA strands DNA:RNA hybrids are usually less stable than RNA:RNA duplexes and, in some cases, even less stable than DNA:DNA duplexes. Available experimental data attributes the relatively lowered stability of DNA:RNA hybrids largely to its intermediate conformational nature between DNA:DNA (B-family) and RNA:RNA (A-family) duplexes. The overall thermodynamic stability of nucleic acid duplexes may originate from several factors including the conformation of backbone, base-pairing and stacking interactions. While it is difficult to ascertain the individual thermodynamic contributions to the overall stabilization of the duplex, it is reasonable to argue that the major factors that promote increased stability of hybrid duplexes are better stacking interactions (electrostatic π-π interactions) and more favorable groove dimensions for hydration. The C2′-S-methyl substitution has been shown to destabilize the hybrid duplex. The notable differences in the rise values among the three hybrids may offer some explanation. While the 2′-S-methyl group has a strong influence on decreasing the base-stacking through high rise values (˜3.2 Å), the 2′-O-methyl group makes the overall structure more compact with a rise value that is equal to that of A-form duplexes (˜2.6 Å). Despite its overall A-like structural features, the SMe_DNA:RNA hybrid structure possesses an average rise value of 3.2 Å which is quite close to that of B-family duplexes. In fact, some local base-steps (CG steps) may be observed to have unusually high rise values (as high as 4.5Å). Thus, the greater destabilization of 2′-S-methyl substituted DNA:RNA hybrids may be partly attributed to poor stacking interactions.

It has been postulated that RNase H binds to the minor groove of RNA:DNA hybrid complexes, requiring an intermediate minor groove width between ideal A- and B-form geometries to optimize interactions between the sugar phosphate backbone atoms and RNase H. A close inspection of the averaged structures for the hybrid duplexes using computer simulations reveals significant variation in the minor groove width dimensions as shown in Table 3. Whereas the O-methyl substitution leads to a slight expansion of the minor groove width when compared to the standard DNA:RNA complex, the S-methyl substitution leads to a general contraction (approximately 0.9 Å). These changes are most likely due to the preferred sugar puckering noted for the antisense strands which induce either A- or B-like single strand conformations. In addition to minor groove variations, the results also point to potential differences in the steric makeup of the minor groove. The O-methyl group points into the minor groove while the S-methyl is directed away towards the major groove. Essentially, the S-methyl group has flipped through the bases into the major groove as a consequence of C2′-endo puckering.

TABLE 11
Minor groove widths averaged over the last 500 ps of simulation time

| Phosphate | | | | DNA:RNA | RNA:RNA |
|---|---|---|---|---|---|
| Distance | DNA:RNA | OMe_DNA:RNA | Sme_DNA:RNA | (B-form) | (A-form) |
| P5-P20 | 15.27 | 16.82 | 13.73 | 14.19 | 17.32 |
| P6-P19 | 15.52 | 16.79 | 15.73 | 12.66 | 17.12 |
| P7-P18 | 15.19 | 16.40 | 14.08 | 11.10 | 16.60 |
| P8-P17 | 15.07 | 16.12 | 14.00 | 10.98 | 16.14 |
| P9-P16 | 15.29 | 16.25 | 14.98 | 11.65 | 16.93 |
| P10-P15 | 15.37 | 16.57 | 13.92 | 14.05 | 17.69 |

Unless otherwise defined herein, alkyl means C₁-C₁₂, C₁-C₈, or C₁-C₆, straight or (where possible) branched chain aliphatic hydrocarbyl.

Unless otherwise defined herein, heteroalkyl means C₁-C₁₂, C₁-C₈, or C₁-C₆, straight or (where possible) branched chain aliphatic hydrocarbyl containing at least one or about 1 to about 3, hetero atoms in the chain, including the terminal portion of the chain. Suitable heteroatoms include N, O and S.

Unless otherwise defined herein, cycloalkyl means C₃-C₁₂, C₃-C₈, or C₃-C₆, aliphatic hydrocarbyl ring.

Unless otherwise defined herein, alkenyl means C₂-C₁₂, C₂-C₈, or C₂-C₆ alkenyl, which may be straight or (where possible) branched hydrocarbyl moiety, which contains at least one carbon-carbon double bond.

Unless otherwise defined herein, alkynyl means C₂-C₁₂, C₂-C₈, or C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. The number of carbon atoms can vary from 1 to about 12, from 1 to about 6, and the total number of ring members varies from three to about 15, or from about 3 to about 8. Suitable ring heteroatoms are N, O and S. Suitable heterocycloalkyl groups include, but are not limited to, morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Suitable aryl rings have about 6 to about 20 ring carbons. In addition, suitable aryl rings include phenyl, napthyl, anthracenyl, and phenanthrenyl.

Unless otherwise defined herein, hetaryl means a ring moiety containing at least one fully unsaturated ring, the ring consisting of carbon and non-carbon atoms. The ring system can contain about 1 to about 4 rings. The number of carbon atoms can vary from 1 to about 12, from 1 to about 6, and the total number of ring members varies from three to about 15, or from about 3 to about 8. Suitable ring heteroatoms are N, O and S. Suitable hetaryl moieties include, but are not limited to, pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the sub-moieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Suitable halo (halogen) substituents are Cl, Br, and I.

The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc.

In all the preceding formulae, the squiggle (˜) indicates a bond to an oxygen or sulfur of the 5′-phosphate. Phosphate protecting groups include those described in U.S. Pat. No. 5,760,209, U.S. Pat. No. 5,614,621, U.S. Pat. No.6,051,699, U.S. Pat. No. 6,020,475, U.S. Pat. No. 6,326,478, U.S. Pat. No. 6,169,177, U.S. Pat. No. 6,121,437, U.S. Pat. No. 6,465,628 each of which is expressly incorporated herein by reference in its entirety.

Oligomerization of modified and unmodified nucleosides is performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The present invention is also useful for the preparation of oligomeric compounds incorporating at least one 2′-O-protected nucleoside. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound. All 2′-O-protecting groups amenable to the synthesis of oligomeric compounds are included in the present invention. In general a protected nucleoside is attached to a solid support by for example a succinate linker. Then the oligonucleotide is elongated by repeated cycles of deprotecting the 5′-terminal hydroxyl group, coupling of a further nucleoside unit, capping and oxidation (alternatively sulfurization). In a more frequently used method of synthesis the completed oligonucleotide is cleaved from the solid support with the removal of phosphate protecting groups and exocyclic amino protecting groups by treatment with an ammonia solution. Then a further deprotection step is normally required for removal of the more specialized protecting groups used for the protection of 2′-hydroxyl groups thereby affording the fully deprotected oligonucleotide.

A large number of 2′-O-protecting groups have been used for the synthesis of oligoribonucleotides but over the years more effective groups have been discovered. The key to an effective 2′-O-protecting group is that it is capable of selectively being introduced at the 2′-O-position and that it can be removed easily after synthesis without the formation of unwanted side products. The protecting group also needs to be inert to the normal deprotecting, coupling, and capping steps required for oligoribonucleotide synthesis. Some of the protecting groups used initially for oligoribonucleotide synthesis included tetrahydropyran-1-yl and 4-methoxytetrahydropyran-4-yl. These two groups are not compatible with all 5′-O-protecting groups so modified versions were used with 5′-DMT groups such as 1-(2-fluorophenyl)-4-methoxypiperidin-4-yl (Fpmp). Reese has identified a number of piperidine derivatives (like Fpmp) that are useful in the synthesis of oligoribonucleotides including 1-((chloro-4-methyl)phenyl)-4′-methoxypiperidin-4-yl (Reese et al., Tetrahedron Lett., 1986, (27), 2291). Another approach was to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group initially used for the synthesis of oligoribonucleotides was the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762). The 2′-O-protecting groups can require special reagents for their removal such as for example the t-butyldimethylsilyl group is normally removed after all other cleaving/deprotecting steps by treatment of the oligomeric compound with tetrabutylammonium fluoride (TBAF).

One group of researchers examined a number of 2′-O-protecting groups (Pitsch, S., Chimia, 2001, (55), 320-324.) The group examined fluoride labile and photolabile protecting groups that are removed using moderate conditions. One photolabile group that was examined was the (2-(nitrobenzyl)oxy)methyl (nbm) protecting group (Schwartz et al., Bioorg. Med. Chem. Lett., 1992, (2), 1019.) Other groups examined included a number structurally related formaldehyde acetal-derived, 2′-O-protecting groups. Also prepared were a number of related protecting groups for preparing 2′-O-alkylated nucleoside phosphoramidites including 2′-O-((triisopropylsilyl)oxy)methyl (2′-O—CH₂—O—Si(iPr)₃, TOM). One 2′-O-protecting group that was prepared to be used orthogonally to the TOM group was 2′-O—((R)-1-(2-nitrophenyl)ethyloxy)methyl) ((R)-mnbm).

Another strategy using a fluoride labile 5′-O-protecting group (non-acid labile) and an acid labile 2′-O-protecting group has been reported (Scaringe, Stephen A., Methods, 2001, (23) 206-217). A number of possible silyl ethers were examined for 5′-O-protection and a number of acetals and orthoesters were examined for 2′-O-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

Although a lot of research has focused on the synthesis of oligoribonucleotides the main RNA synthesis strategies that are presently being used commercially include 5′-O-DMT-2′-O-t-butyldimethylsilyl (TBDMS), 5′-O-DMT-2′-O-(1(2-fluorophenyl)-4-methoxypiperidin-4-yl) (FPMP), 2′-O-((triisopropylsilyl)oxy)methyl(2′-O—CH₂-O—Si(iPr)₃ (TOM), and the 5′-O-silyl ether-2′-ACE(5′-O-bis(trimethylsiloxy)cyclododecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention.

The primary groups being used for commercial RNA synthesis are:

| | |
|---|---|
| TBDMS = | 5′-O-DMT-2′-O-t-butyldimethylsilyl; |
| TOM = | 2′-O-((triisopropylsilyl)oxy)methyl; |
| DOD/ | (5′-O-bis(trimethylsiloxy)cyclododecyloxysilyl ether-2′- |
| ACE = | O-bis(2-acetoxyethoxy)methyl |
| FPMP = | 5′-O-DMT-2′-O-(1(2-fluorophenyl)-4-methoxypiperidin-4-yl). |

All of the aforementioned RNA synthesis strategies are amenable to the present invention. Strategies that would be a hybrid of the above e.g. using a 5′-protecting group from one strategy with a 2′-O-protecting from another strategy is also amenable to the present invention.

The preparation of ribonucleotides and oligomeric compounds having at least one ribonucleoside incorporated and all the possible configurations falling in between these two extremes are encompassed by the present invention. The corresponding oligomeric comounds can be hybridized to further oligomeric compounds including oligoribonucleotides having regions of complementarity to form double-stranded (duplexed) oligomeric compounds. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The methods of preparing oligomeric compounds of the present invention can also be applied in the areas of drug discovery and target validation. The present invention comprehends the use of the oligomeric compounds and suitable targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity is evaluated according to existing literature (Elbashir et al., Nature (2001), 411, 494-498; Nishikura et al., Cell (2001), 107, 415-416; and Bass et al., Cell (2000), 101, 235-238.)

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Animals

Balb/c mice, 18-24 g (5-7 weeks old), were obtained from Charles River (Wilmington Mass.) and used for subsequent in vitro and in vivo experiments. Animals were housed in polycarbonate cages and given access to chow and water ad libitum, in accordance with protocols approved by the Institutional Animal Care and Use Committee.

### Example 2

### Oligonucleotides

All chimeric MOE Gapmers are 20-mer phosphorothioate oligodeoxynucleotides containing 2′-O-methoxyethyl (2′-MOE) modifications at positions 1-5 and 16-20. MOE Gapmers and the 2′-deoxy unmodified phosphorothioate oligodeoxynucleotides ODN-PTEN and ODN-PTEN(6 MM) were synthesized on a Milligen model 8800 DNA synthesizer (Millipore Inc., Bedford Mass.) using conventional solid-phase triester chemistry (Sanghvi, 1999) at Isis Pharmaceuticals Inc. Deprotected and desalted siRNA analogs were obtained from Dharmacon Research, Inc. (LaFayette, Colo.). Sequences of siRNA compounds, and the oligonucleotides and placement of their 2′-O-methoxyethyl modifications, are detailed in Table 12. 2′-MOE Gapmers (MG) are first generation 20-mer phosphorothioate oligodeoxynucleotides which contain 2′-O-methoxyethyl (2′-MOE) modifications at positions 1-5 and 16-20 (boldface type). ISIS 160847 and 160848 are first generation phosphorothioate oligodeoxynucleotides (ODN). Both antisense and sense strands are shown for each siRNA construct (si). Six-base mismatch (6 MM) control oligonucleotides are of similar nucleoside composition as the respective antisense oligonucleotides.

TABLE 12

| | | | | | |
|---|---|---|---|---|---|
| | | | Sequence | SEQ ID | |
| Isis No. | Target | Strand | (5′ to 3′) | NO: | Composition |

| | | | | | | |
|---|---|---|---|---|---|---|
| ASOs | | | | | | |
| | 160847 | PTEN | as | CTGCTAGCCTCTGGATTT | 16 | 2′-deoxy P=S; 5- |
| | | | | GA | | MeC |
| ODN- | 160848 | PTEN | as | CTTCTGGCATCCGGTTTA | 17 | 2′-deoxy P=S; 5- |
| PTEN | | | (6MM) | GA | | MeC |
| (6MM) | | | | | | |
| MG-PTEN | 116847 | PTEN | as | CTGCTAGCCTCTGGATTT | 18 | 5_10_5 2′MOE |
| | | | | GA | | Gapmer; 5-MeC |
| MG-PTEN | 116848 | PTEN | as | CTTCTGGCATCCGGTTTA | 19 | 5_10_5 2+MOE |
| (6MM) | | | (6MM) | GA | | Gapmer; 5-MeC |
| MG-Fas | 22023 | Fas | as | TCCAGCACTTTCTTTTCC | 20 | 5_10_5 2′MOE |
| | | | | GG | | Gapmer; 5-MeC |
| MG-Fas | 29836 | Fas | as | TCCATCTCCTTTTATGCC | 21 | 5_10_5 2′MOE |
| (6MM) | | | (6MM) | GG | | Gapmer; 5-MeC |
| MG-MTTP | 144477 | MTTP | as | CCCAGCACCTGGTTTGCC | 22 | 5_10_5 2′MOE |
| | | | | GT | | Gapmer; 5-MeC |
| MG-ApoB | 147764 | Apo B | as | GTCCCTGAAGATGTCAA | 23 | 5_10_5 2′MOE |
| | | | | TGC | | Gapmer; 5-MeC |
| siRNAs † | | | | | | |
| si-PTEN | 263186 | PTEN | as | CU*GC*UA*GC*CU*CU*G | 24 | alt *P=S, P=O |
| | | | | G*AU*UU*GdT*dT | | linkage; 3′-dTdT |
| | | | | | | overhang |
| | 263187 | PTEN | s | CA*AA*UC*CA*GA*GG*C | 25 | alt *P=S, P=O |
| | | | | U*AG*CA*GdT*dT | | linkage; 3′-dTdT |
| | | | | | | overhang |
| si-PTEN | 263188 | PTEN | as | CU*UC*UG*GC*AU*CC*G | 26 | alt *P=S, P=O |
| (6MM) | | | (6MM) | G*UU*UA*GdT*dT | | linkage; 3′-dTdT |
| | | | | | | overhang |
| | 263189 | PTEN | s | CU*AA*AC*CG*GA*UG*C | 27 | alt *P=S, P=O |
| | | | (6MM) | C*AG*AA*GdT*dT | | linkage; 3′-dTdT |
| | | | | | | overhang |
| si-PTEN | 278626 | PTEN | as | CUGCUAGCCUCUGGAUU | 28 | unmodified RNA |
| (blunt) | | | | UGAC | | |
| | 278627 | PTEN | s | GUCAAAUCCAGAGGCUA | 29 | unmodified RNA |
| | | | | GCAG | | |
| si-Fas ‡ | — | Fas | as | 5′-P | 30 | unmodified RNA; |
| | | | | GUCUGGUUUGCACUUGC | | 5′-Phosphate, 3′- |
| | | | | ACdTdT | | dTdT overhang |
| | — | Fas | s | 5′-P | 31 | unmodified RNA; |
| | | | | GUGCAAGUGCAAACCAG | | 5′-Phosphate, 3′- |
| | | | | ACdTdT | | dTdT overhang |
| si-Fas | 328798 | Fas | as | 5′-P | 32 | unmodified RNA; |
| (6MM) | | | (6MM) | GUGUCGUGUUCAGUUCC | | 5′-Phosphate, 3′- |
| | | | | ACdTdT | | dTdT overhang |
| | 328799 | Fas | s | 5′-P | 33 | unmodified RNA; |
| | | | (6MM) | GUGGAACUGAACACGAC | | 5′-Phosphate, 3′- |
| | | | | ACdTdT | | dTdT overhang |

† siRNAs are named as dsRNA sets (e.g. si-PTEN includes the antisense strand 263186 and sense strand 263187)
‡ si-Fas sequences from Song et. al. (2003)
Legend: as—antisense strand, s—sense strand, ApoB—Apolipoprotein B, PTEN—Phosphotase and Tensin homolog deleted on chromosome Ten, MTTP—Microsomal Triglyceride Transfer Protein

### Example 3

### In Vitro Analysis

Primary hepatocyte isolation/culture. Mouse hepatocytes were isolated from mice using a two step in situ liver perfusion as previously described (McQueen et al., Cell. Biol. Toxicol., 1989, 5, 201-206). Briefly, animals were anesthetized with Avertin (50 mg/kg, intraperitoneal) and the portal vein was exposed. Hank's Balanced Salt Solution (Life Technologies, Grand Island, N.Y.) was perfused through the portal vein for 3.5 min at 2 ml/min followed by Williams Medium E (WME: Life Technologies, Grand Island, N.Y.) containing 0.3 mg/ml collagenase B (Roche Molecular Biochemicals, Indianapolis, Ind.) for 5.5 minutes. The liver was removed from the animal and gently massaged through Nitex nylon mesh (Tetko, Depew, N.Y.) to obtain a suspension of cells. The suspension was centrifuged (4 minutes at 500 rpm) and the supernatant discarded. The remaining pellet was gently resuspended in WME and centrifuged (4 minutes at 500 rpm) two more times to remove nonparenchymal cells. The pelleted hepatocytes were resuspended in WME supplemented with 10% fetal bovine serum (FBS)(v:v) and the concentration of cells was determined. For plating, cells were resuspended to the desired working concentration in WME supplemented with 10% FBS, 1% L-glutamine (v:v), 1% HEPES (v:v), and 1% gentamycin (antimitotic-antibiotic). Cells were plated on Primaria™ coated 6-well plates at a density of 100,000 per ml or Primaria™ 96-well coated plates at a density of 10,000 per ml. Cells were allowed to adhere to plates for one hour and then gently washed with PBS to remove dead cells and the media replaced with fresh HepatoZYME™ media (Invtirogen, Carlsbad, Calif.) supplemented with 1% L-glutamine (v:v), 1% HEPES (v:v), 1% non-essential amino acids (NEAA) and 1% gentamycin (antimitotic-antibiotic).

In vitro hepatocyte oligodeoxynucleotide transfections. For experiments transfecting primary hepatocytes with cationic lipids, transfections were performed either four hours after plating or after an additional 8-12 hours (overnight). No difference in transfection results were observed comparing the two plating intervals (data not shown). The oligonucleotide or siRNA (oligo/siRNA) was mixed with Lipofectin (Invitrogen, Carlsbad, Calif.) at a working concentration of 3 μg per 100 nM of single strand DNA or RNA per 1 ml of media. Prior to addition to cells, the mix was incubated 5-10 minutes as per vendor recommendations. Plating media was then removed and the cells were treated for 4-6 hours, the media changed to fresh HepatoZYME™ (supplemented as above) and cells incubated overnight for an additional 16-20 hours. Cells were then lysed and the RNA isolated and purified as described below. For free uptake studies, cells were allowed to adhere to the plastic for 4 hours then treated with the oligos/siRNA in the HepatoZYME™ media for 12-16 hours (overnight). Cells were then lysed and the RNA isolated and purified as described below.

RNA isolation and expression analysis. In vitro total RNA was harvested at the indicated times post-transfection using the RNeasy Mini kit (Qiagen, Valencia, Calif.) for the 6-well treatments and using the Qiagen BioRobot 3000 for the 96-well plates, according to the manufacturers protocol. Gene expression was determined via real time quantitative RT-PCR on the ABI Prism 7700 system (Applied Biosystems, Foster City Calif.) as suggested by the manufacturer and described in the literature (Gibson et al., Genome Res., 1996, 6, 995-1001; Winer et al., Anal. Biochem., 1999, 270, 41-49; and Vickers et al., J. Biol. Chem., 2003, 278, 7108-7118). Primers and probes were obtained from IDT, Inc. (Coralville, Iowa) and the following primer/probe sets were used: PTEN (accession number U92437.1), forward primer (ATGACAATCATGTTGCAGCAATTC; SEQ ID NO:1), reverse primer (CGATGCAATAAATATGCACAAATCA; SEQ ID NO:2), and probe (GTAAAGCTGGAAAGGGACGGACTGGT; SEQ ID NO:3); Fas (accession number M83649.1), forward primer (TCCAAGACACAGCTGAGCAGA; SEQ ID NO:4), reverse primer (TGCATCACTCTTCCCATGAGAT; SEQ ID NO:5), and probe (AGTCCAGCTGCTCCTGTGCTGGTACC; SEQ ID NO:6); Apolipoprotein B (accession number M35186.1), forward primer (CGTGGGCTCCAGCATTCTA; SEQ ID NO:7), reverse primer (AGTCATTTCTGCCTTTGCGTC; SEQ ID NO:8), and probe (CCAATGGTCGGGCACTGCTCAA; SEQ ID NO:9); Microsomal triglyceride transfer protein (accession number NM_{—}008642.1), forward primer (GAGCGGTCTGGATTTACAACG; SEQ ID NO:10), reverse primer (AGGTAGTGACAGATGTGGCTTTTG; SEQ ID NO:11), and probe (CAAACCAGGTGCTGGGCGTCAGT; SEQ ID NO:12); and murine cyclophilin A (accession number), forward primer (TCGCCGCTTGCTGCA; SEQ ID NO:13), reverse primer (ATCGGCCGTGATGTCGA; SEQ ID NO:14) and probe (CCATGGTCAACCCCACCGTGTTC; SEQ ID NO:15). Cyclophilin A mRNA levels were used with 96-weel transfection experiments as an internal standard for sample to sample normalization. All mRNA expression levels were normalized both to RiboGreen (Molecular Probes, Eugene, Oreg.), and GAPDH mRNA, also determined by quantitave RT-PCR (data not shown), from the same total RNA samples. Dose-response trends were independent of the normalization technique, and only RiboGreen normalized data is presented here.

Statistical Analysis. Simple Student's T-Test were performed.

Primary hepatocyte monolayer model. Mouse primary hepatocytes plated in 6-well plates were dosed with ISIS 116847(MG-PTEN), a MOE gapmer specific for PTEN (Butler et al., Diabetes, 2002, 51, 1028-1034), at 25 and 100 nM in the presence of lipofectin. PTEN mRNA expression levels fell in a dose-dependent manner. Transcript expression was reduced by a maximum of 87% (0.13±0.06 of control) at 100 nM, with an IC50 of approximately 25 nM. Doses above 100 nM did not significantly decrease message knockdown (data not shown).

### Example 4

### In Vivo Analysis

In vivo oligonucleotide treatment. MOE gapmer or siRNA oligonucleotides were administered in saline (0.9% NaCl) via intravenous tail vein injection at the indicated doses once per day for five days. Mice were sacrificed on day five, six hours post administration. Liver RNA was isolated as described below.

RNA isolation and expression analysis. Total RNA was extracted from mouse liver by homogenizing liver in guanidinium isothiocyanate at time of sacrifice, and isolating total RNA standard cesium chloride gradient centrifugation techniques. Gene expression was determined via real time quantitative RT-PCR on the ABI Prism 7700 system (Applied Biosystems, Foster City Calif.) as described above.

Primary hepatocyte monolayer model. Mouse primary hepatocytes plated in 6-well plates were dosed with ISIS 116847(MG-PTEN), a MOE gapmer specific for PTEN (Butler et al., Diabetes, 2002, 51, 1028-1034), at 25 and 100 nM in the presence of lipofectin PTEN mRNA expression levels fell in a dose-dependent manner. Transcript expression was reduced by a maximum of 87% (0.13±0.06 of control) at 100 nM, with an IC50 of approximately 25 nM. Doses above 100 nM did not significantly decrease message knockdown (data not shown).

### Example 5

### Design of Single and Double-strand Antisense Constructs

MG-PTEN is a 20-base chimeric 2′-O-methoxyethyl oligonucleotide (MOE gapmer) that has previously been demonstrated to be a potent inhibitor of mouse PTEN expression (Butler et al., Diabetes, 2002, 51, 1028-1034). siRNA analogs to the same coding region targeted by MG-PTEN were synthesized to compare the in vitro dose-response characteristics of the two classes of antisense compounds. Table 12 is a representative list of oligonucleotides used, their sequences, and specific chemical modifications.

Single-strand MOE gapmers and double-strand RNA (dsRNA) show comparable activity profiles in primary mouse hepatocytes under cationic lipid transfection conditions.

Mouse primary hepatocytes plated in 96-well plates were transfected with either: the MOE gapmer MG-PTEN, the 6-base mismatch (MG-PTEN(6 MM)), the blunt ended dsRNA analog to the MOE 116847 site (si-PTEN(blunt)), the 2-nt 3′-overhang dsRNA analog with mixed backbone (si-PTEN), or the 6 base-pair 2-nt 3′-overhang dsRNA mismatch to si-PTEN with mixed backbone (si-PTEN(6 MM)) in the presence of Lipofectin. Both the MOE gapmer and the dsRNA designed against the target region 116847 significantly reduced PTEN mRNA in a dose-dependent manner. The mixed backbone dsRNA containing 2-nt 3′-dTdT overhangs, si-PTEN, appeared to have a slightly lower IC50 than the corresponding blunt-end dsRNA, si-PTEN (blunt). While, the IC50 for the 2′-MOE MG-PTEN was significantly lower (12.5 nM), maximal mRNA reduction was achieved at 200 nM (higher dosages not shown). The lower IC50 observed for MG-PTEN could reflect mechanistic differences in target reduction or reflect that the sequence used for comparative purposes was optimized for MOE gapmer chemistry. The PTEN mismatch control to the mixed backbone dsRNA containing 2-nt 3′-dTdT overhangs, si-PTEN(6 MM), did not effect PTEN mRNA levels, suggesting that target reduction was not due to non-specific dsRNA or siRNA effects.

Uptake activity is independent of sequence. Evidence suggests that the uptake of antisense oligonucleotides is independent of oligonucleotide sequence (Leeds et al., Nucleosides Nucleotides, 1997, 16, 1689-1693; and Geary et al., J. Pharmacol. Exp., 2001, 296, 890-897). To confirm that the in vitro Lipofectin mediated dose-dependent inhibition of target observed with the MOE gapmer MG-PTEN could be reproduced with other potent antisense MOE gapmers, another potent MOE gapmer, MG-Fas, was selected for Lipofectin mediated dose-response analysis. MG-Fas targets a sequence within the translated region of the murine Fas transcript. It is a 20-base chimeric MOE gapmer that has been shown to inhibit Fas expression both in vitro and in vivo in a dose-dependent and sequence-specific manner. It has been reported that both Fas mRNA and protein levels fall as much as 90% in mice dosed with MG-Fas (Zhang et al., Nat. Biotechnol., 2000, 18, 862-867). As described for PTEN, mouse primary hepatocytes were plated in 96-well plates and transfected with either: the MOE gapmer MG-Fas; MG-Fas(6 MM), a 6 base mismatch control to MG-Fas; si-Fas, a dsRNA containing an antisense strand using the anti-Fas siRNA sequence 1 from a study by Song et. al. (Nat. Med., 2003, 9, 347-351), where they reported that hydrodynamic tail vein injection of this sequence into mice reduced Fas mRNA expression in liver hepatocytes by approximately 86% of control; and si-Fas(6 MM), a 6 base mismatch control dsRNA. Both MG-Fas and si-Fas reduced Fas mRNA expression in a dose-dependent manner, MG-Fas reducing Fas mRNA to 0.76±0.12 and 0.04±0.03 of control at 75 and 300 nM, respectively; and si-Fas reducing expression to 0.82±0.05 and 0.29±0.08 of control at 75 and 300 nM, respectively. Thus, the PTEN and Fas data taken together suggest that both MOE gapmers and dsRNAs inhibit gene expression in a dose-dependent manner when transfected into isolated mouse hepatocytes.

To further investigate whether chemical modifications to the MOE gapmer backbone would alter dose-response characteristics, mouse primary hepatocytes were transfected with either the MOE gapmer MG-PTEN; MG-PTEN(6 MM), the 6 base mismatch control to MG-PTEN; ODN-PTEN, a first generation unmodified 20-mer phosphorothioate (PS) oligonucleotide (no MOE modifications); or ODN-PTEN(6 MM), ODN-PTEN's 6 base mismatch control (Table 12). These oligodeoxynucleotides contain PS backbones, but are uniformly 2′-OH unmodified. Both the MOE gapmer and the unmodified PS oligonucleotide significantly reduced PTEN mRNA in a dose-sensitive manner. The slightly greater target knockdown seen with the MG-PTEN supports previous observations that MOE modified PS oligonucleotides have slightly increased binding affinity for their complementary RNAs (Crooke et al., Biochem. J., 1995, 312(Pt 2), 599-608). MOE gapmer increased nuclease resistance may also be increasing MG-PTENs efficacy in this assay by increasing intracellular concentrations relative to ODN-PTEN over time.

Single-strand MOE gapmers and PS oligonucleotides show different activity profiles than double-strand RNA (dsRNA) in primary mouse hepatocytes in free-uptake conditions. Graham et al. (J. Pharmacol. Exp. Therap., 1998, 286, 447-458) has previously demonstrated both free uptake and activity of MOE gapmers incubated with primary hepatocytes without the use of cationic lipids similar to that seen in vivo. To investigate the dose-response sensitivity of the MOE gapmers, experiments were conducted in mouse primary hepatocytes plated in 6-well plates with both MG-PTEN and MG-Fas at concentrations ranging from 75 to 10000 nM (see above procedures). The expression levels of both targeted PTEN and Fas mRNA subsequently fell in a dose-dependent manner. Maximal inhibition (˜90%) of both PTEN and Fas mRNA levels was achieved at 3000 nM, with an IC50 of approximately 350 nM for PTEN and 750 nM for Fas. Higher concentrations of either MG-PTEN or MG-Fas did not significantly reduce transcript levels. Six base mismatches to both MG-PTEN and MG-Fas, MG-PTEN(6 MM) and MG-Fas(6 MM), did not reduce transcript levels; arguing against ASO dose related mRNA toxicity (data not shown). PTEN and Fas transcript levels were normalized with RiboGreen. However, the dose-response trends observed were independent of the normalization technique, as normalization using either RiboGreen or GAPDH transcript levels yielded similar results (GAPDH data not shown).

To further confirm that the in vitro dose-dependent inhibition of target observed with both MG-PTEN and MG-Fas could be reproduced with other potent antisense MOE gapmers, two additional potent MOE gapmers, MG-ApoB and MG-MTTP were selected for free uptake dose-response analysis (see Table 12). MG-ApoB is a potent inhibitor of the mouse apolipoprotein B (ApoB) (unpublished data). MG-MTTP targets mouse microsomal triglyceride transfer protein (MTTP) (unpublished data). All MOE gapmers tested displayed similar dose-response dynamics (data not shown), suggesting that the mechanism of MOE gapmer uptake and subsequent RNA inhibition is highly conserved.

To determine whether dsRNA might also demonstrate both free uptake and activity without the use of cationic lipids, mouse primary hepatocytes were dosed with either si-PTEN(blunt), si-PTEN, si-PTEN(6 MM), the MG-PTEN, MG-Fas, MG-Fas(6 MM), si-Fas, or si-Fas(6 MM); at concentrations ranging from 375 to 1500 nM. The siRNA constructs are capable of specific target reduction in the presence of the cationic lipid Lipofectin. However, whereas the MOE modified single-strand DNAs MG-PTEN and MG-Fas show robust target reduction even at the lowest concentrations (375 nM), no target reduction was observed with dsRNA under these conditions.

Given the lack of activity observed for dsRNA in free uptake experiments under the expressed conditions, it was of interest to determine whether MOE modifications were aiding free uptake of single-strand DNA. Again, the unmodified homologs to MG-PTEN and the 6 base mismatch MG-PTEN(6 MM), ODN-PTEN and ODN-PTEN(6 MM) were used. These first generation, unmodified molecules demonstrate a dose responsive, specific target reduction. Again, the MOE modified gapmer MG-PTEN demonstrated much greater message knockout, suggesting that the MOE modification may assist and improve the oligonucleotide delivery in the absence of transfection reagents. Again, the half-lives of unmodified first generation oligodeoxynucleotides are much shorter, which may in part explain the reduced activity observed with these molecules.

Capillary gel electrophoresis (CGE) was used to look at the stability of the duplex siRNA constructs in the treatment media (see above for media description) at different time points. If the duplex is still intact after 16 hrs, which is the duration of our treatments, the construct is considered valid for the in vitro assay proposed herein, and tested for uptake and/or activity.

### Example 6

### In vivo Target Inhibition—MOE gapmers versus dsRNAs

Given the observed robust target inhibition with both single-stranded MOE gapmers, unmodified PS oligonucleotides and dsRNA when using Lipofectin as a transfection agent, but no observation of dsRNA activity when a transfection reagent was not used under the conditions described above, coupled with reports that dsRNA when administered in vivo via high pressure tail injections knock down target (McCafferey et al., Nature, 2002, 418, 38-39; Lewis et al., Nat. Genet., 2002, 32, 107-108; and Song et al., Nat. Med., 2003, 3, 347-351), it was of interest to compare MOE gapmer and dsRNA activity in vivo using conventional intravenous injections. MG-PTEN, MG-PTEN(6 MM), si-PTEN and si-PTEN(6 MM) were administered daily for 5 days at concentrations of either 2.5 mg/kg or 25 mg/kg. Only MG-PTEN reduced PTEN mRNA levels in liver. Further, in a separate study, animals were dosed daily for five days with si-PTEN(blunt) to concentrations as high as 50 mg/kg. Again, only MG-PTEN reduced PTEN mRNA levels in liver. No effect was observed for intraperitoneal injected si-PTEN(blunt) under the conditions described above. High-pressure delivery systems may mimic in vitro transfection mediated oligonucleotide delivery by altering cell membrane permeability; however, we are unaware of any studies demonstrating mRNA knockdown with dsRNA using conventional delivery systems.

The results suggest that the in vitro primary hepatocyte model correlates both with single-strand DNA oligonucleotide (both MOE gapmer and PS oligonucleotides) and dsRNA in vivo activity. Specifically, whereas single-strand oligonucleotides effectively decrease target mRNA expression both in vitro and in vivo without the aid of a delivery system, dsRNA does not decrease target mRNA expression in hepatocytes in vitro without the aid of transfection reagents under the conditions described above or in vivo when delivered by conventional dosing methods.

### Example 7

### Sequential Delivery

dsRNA is now shown to decrease target mRNA expression in hepatocytes in vitro without the aid of transfection reagents. The following PTEN oligomeric compounds were used in a free uptake assay in primary mouse hepatocytes, as desribed above: Compound 303912 antisense (TTTGTCTCTGGTCCTTACTT; SEQ ID NO:34; PS backbone, RNA sugar); Compound 316449 antisense (TTTGTCTCTGGTCCTTACTT; SEQ ID NO:35; PS backbone, 3′ 3x Ome sugar); Compound 347849 antisense (TTTGTCTCTGGTCCTTACTTT; SEQ ID NO:36; PO backbone, RNA sugar); Compound 341315 sense (AAGTAAGGACCAGAGACAAA; SEQ ID NO:37; PS backbone, full Ome sugar); and Compound 308746 sense (AAGTAAGGACCAGAGACAAA; SEQ ID NO:38; PO backbone, RNA sugar). The mRNA target levels were normalized to total RNA

(RiboGreen). The results are expressed as % UTC and are reported in Table 13. The following Table 2:shows the delivery protocol and results of treatment of each group:

TABLE 13

| | 0 | 1 | 2 | 4 | % UTC |
|---|---|---|---|---|---|
| A | 303912 | | | add 341315 (4 uM) | 130 |
| B | 303912 | replace with 341315 | (replaced with 303912 instead by mistake) | | 105 |
| C | 303912 | | replace with 341315 | | 104 |
| D | 303912 | | | replace with 341315 | 94 |
| E | 316449 | | | add 341315 (4 uM) | 141 |
| F | 316449 | replace with 341315 | | | 103 |
| G | 316449 | | replace with 341315 | | 107 |
| H | 316449 | | | replace with 341315 | 114 |
| I | 341315 | | | add 303912 (4 uM) | 31 |
| J | 341315 | replace with 303912 | | | 122 |
| K | 341315 | | replace with 303912 | | 23 |
| L | 341315 | | | replace with 303912 | 29 |
| M | 341315 | | | add 316449 (4 uM) | 50 |
| N | 341315 | replace with 316449 | | | 120 |
| O | 341315 | | replace with 316449 | | 42 |
| P | 341315 | | | replace with 316449 | 50 |
| Q | 303912:341315 | | | | 31 |
| R | 316449:341315 | | | | 68 |
| S | 303912 | | | | 82 |
| T | 316449 | | | | 89 |
| U | 347849:308746 | | | | 83 |

A, E, I & M - cells were treated at time 0 with one strand; after four hours, the other strand was added.
B, F, J & N - cells were treated at time 0 with one strand; after 1 hour, the first treatment is removed and replaced with the one containing the other strand.
C, G, K & O - cells were treated at time 0 with one strand; after 2 hours, the first treatment is removed and replaced with the one containing the other strand.
D, H, L & P - cells were treated at time 0 with one strand; after 4 hours, the first treatment is removed and replaced with the one containing the other strand.
Q-U - these were treated at time 0 and left untouched until lysis time.

Oligomeric siRNA compound combinations 303912:341315 and 316449:341315 show activity in this free uptake system. Sequentially treating with these antisense sequences first followed by the sense strand do not show appreciable target reduction. In contrast, sequentially treating with the sense strand first followed by the antisense strand shows good activity except at the one hour strand switching. In addition, sequential treatment with 303912 is as potent as the corresponding siRNA. Further, sequential treatment with 316449 is more potent than the corresponding siRNA. Even further, chemical modification of the terminal three 3′ nucleotides to comprise 2′-Ome sugar residues, as in Compound 316449, is suitable.

A dose-response study was also performed in the primary hepatocyte free uptake assay with the following oligomeric compounds: Compound 303912 antisense (TTTGTCTCTGGTCCTTACTT; SEQ ID NO:34; PS backbone, RNA sugar); Compound 335449 antisense (TTTGTCTCTGGTCCTTACTT; SEQ ID NO:39; PO backbone, RNA sugar); Compound 341315 sense (AAGTAAGGACCAGAGACAAA; SEQ ID NO:37; PS backbone, full 2′-Ome sugar); Compound 330696 sense (AAGTAAGGACCAGAGACAAA; SEQ ID NO: 40; PO backbone, full 2′-Ome sugar); and Compound 344178 sense (AAGTAAGGACCAGAGACAAA; SEQ ID NO:41; PS backbone, RNA sugar). Results are shown in FIG. 1.

### Example 8

### Sequential Delivery-Effects of Modifications

The following PTEN oligomeric compounds were used in a free uptake assay in primary mouse hepatocytes, as desribed above: Compound 303912 antisense (TTTGTCTCTGGTCCTTACTT; SEQ ID NO:34; PS backbone, RNA sugar, 5′phosphate); Compound 335449 antisense (TTTGTCTCTGGTCCTTACTT; SEQ ID NO:39; PO backbone, RNA sugar, 5′phosphate); Compound 317502 antisense (TTTGTCTCTGGTCCTTACTTT; SEQ ID NO:42; PS backbone, RNA sugar with 2′Fluro modifications at the bolded positions, 5′phosphate); Compound 354626 antisense (TTTGTCTCTGGTCCTTACTT; SEQ ID NO:43; PS backbone, RNA sugar, 5′hydroxyl); Compound 116847 antisense (TTTGTCTCTGGTCCTTACTT; SEQ ID NO:44; PS backbone, MOE/DNA sugar having 2′MOE groups at positions 1-5 and 16-20); Compound 344178 sense (AAGTAAGGACCAGAGACAAA; SEQ ID NO:41; PS backbone, RNA sugar); Compound 341315 sense (AAGTAAGGACCAGAGACAAA; SEQ ID NO:37; PS backbone, full 2′O-methyl sugar); Compound 354622 sense (AAGCAACGAGAAGCGATAAA; SEQ ID NO:45; PS backbone, full 2′O-methyl sugar; 6base mismatch to Compound 341315) and Compound 330696 sense (AAGTAAGGACCAGAGACAAA; SEQ ID NO:40; PO backbone, full 2′-O-methyl sugar). The mRNA target levels were normalized to total RNA (RiboGreen). The results are expressed as % UTC and are reported in Table 14. The following Table 3 shows the delivery protocol and results of treatment of each group:

TABLE 14
Free Uptake in mouse hepatocytes-effects of chemical modifications

| | Time (hr) | | | | |
|---|---|---|---|---|---|
| | 0 | 1 | 2 | 4 | % UTC |
| A | 344178 | | | add 303912 (2 uM) | 45 |
| B | 344178 | replace with 303912 | | | 28 |
| C | 344178 | | replace with 303912 | | 41 |
| D | 344178 | | | replace with 303912 | 43 |
| E | 344178 | | | add 317502 (2 uM) | 50 |
| F | 344178 | replace with 317502 | | | 40 |
| G | 344178 | | replace with 317502 | | 66 |
| H | 344178 | | | replace with 317502 | 73 |
| I | 344178 | | | add 335449 (2 uM) | 89 |
| J | 344178 | replace with 335449 | | | 79 |
| K | 344178 | | replace with 335449 | | 82 |
| L | 344178 | | | replace with 335449 | 82 |
| M | 344178 | | | add 354626 (2 uM) | 49 |
| N | 344178 | replace with 354626 | | | 29 |
| O | 344178 | | replace with 354626 | | 45 |
| P | 344178 | | | replace with 354626 | 47 |
| Q | 354622 | | | add 303912 (2 uM) | 98 |
| R | 354622 | replace with 303912 | | | 86 |
| S | 354622 | | replace with 303912 | | 88 |
| T | 354622 | | | replace with 303912 | 94 |
| U | 303912 | | | | 95 |
| V | 317502 | | | | 122 |
| W | 354626 | | | | 96 |
| X | 116847 | | | | 7 |
| Y | 303912:344178 | | | | 23 |
| Z | 354626:344178 | | | | 23 |

A, E, I, M and Q - cells were treated at time 0 with one strand; after four hours, the other strand was added.
B, F, J, N and R - cells were treated at time 0 with one strand; after 1 hour, the first treatment is removed and replaced with the one containing the other strand.
C, G, K, O and S - cells were treated at time 0 with one strand; after 2 hours, the first treatment is removed and replaced with the one containing the other strand.
D, H, L, P and T - cells were treated at time 0 with one strand; after 4 hours, the first treatment is removed and replaced with the one containing the other strand.
U, V, W, X, Y and Z - these were treated at time 0 and left untouched until lysis time.

Oligomeric siRNA compound combinations 303912:344178 and 354626:344178 show activity in this free uptake system. Sequentially treating with the sense strand of these duplexes first followed by the antisense strand shows good activity at every time point tested. In addition, sequential treatment with 303912 or 354626 at the one hour time point is as potent as the corresponding siRNA (compare sequential treatments B and N with siRNA treatments Y and Z). Even further, chemical modification with a fluro group at select 2′ positions in the antisense strand was also found to effectively reduce target RNA levels.

### Example 9

### Sequential Delivery-Comparison of Backbone Chemistry in 2′OMe Background Construct

The following PTEN oligomeric compounds were used in a free uptake assay in primary mouse hepatocytes, as desribed above: Compound 303912 antisense (TTTGTCTCTGGTCCTTACTT; SEQ ID NO:34; PS backbone, RNA sugar, 5′phosphate); Compound 335449 antisense (TTTGTCTCTGGTCCTTACTT; SEQ ID NO:39; PO backbone, RNA sugar, 5′phosphate); Compound 317502 antisense (TTTGTCTCTGGTCCTTACTTT; SEQ ID NO:42; PS backbone, RNA sugar with 2′Fluro modifications at the bolded positions, 5′phosphate); Compound 354626 antisense (TTTGTCTCTGGTCCTTACTT; SEQ ID NO:43; PS backbone, RNA sugar, 5′hydroxyl); Compound 116847 antisense (TTTGTCTCTGGTCCTTACTT; SEQ ID NO:44; PS backbone, MOE/DNA sugar having 2′MOE groups at positions 1-5 and 16-20); Compound 341315 sense (AAGTAAGGACCAGAGACAAA; SEQ ID NO:37; PS backbone, full 2′O-methyl sugar); and Compound 330696 sense (AAGTAAGGACCAGAGACAAA; SEQ ID NO:40; PO backbone, full 2′-O-methyl sugar). The mRNA target levels were normalized to total RNA (RiboGreen). The results are expressed as % UTC and are reported in Table 15. The following Table 15 shows the delivery protocol and results of treatment of each group. Entry of “ND” in the Table indicates at least one reagent or construct was limiting and no data were gathered for this group.

TABLE 15
Free Uptake in mouse hepatocytes-effects of backbone modifications

| | Time (hr) | | | | |
|---|---|---|---|---|---|
| | 0 | 1 | 2 | 4 | % UTC |
| A | 330696 | | | add 303912 (2 μM) | ND |
| B | 330696 | replace with 303912 | | | ND |
| C | 330696 | | replace with 303912 | | ND |
| D | 330696 | | | replace with 303912 | ND |
| E | 330696 | | | add 335449 (2 μM) | 84 |
| F | 330696 | replace with 335449 | | | 86 |
| G | 330696 | | replace with 335449 | | 85 |
| H | 330696 | | | replace with 335449 | 104 |
| I | 341315 | | | add 303912 (2 μM) | ND |
| J | 341315 | replace with 303912 | | | 24 |
| K | 341315 | | replace with 303912 | | 27 |
| L | 341315 | | | replace with 303912 | 32 |
| M | 341315 | | | add 317502 (2 μM) | 79 |
| N | 341315 | replace with 317502 | | | 52 |
| O | 341315 | | replace with 317502 | | 69 |
| P | 341315 | | | replace with 317502 | 97 |
| Q | 341315 | | | add 335449 (2 μM) | 96 |
| R | 341315 | replace with 335449 | | | 89 |
| S | 341315 | | replace with 335449 | | 94 |
| T | 341315 | | | replace with 335449 | 105 |
| U | 341315 | | | add 354626 (2 μM) | 35 |
| V | 341315 | replace with 354626 | | | 29 |
| W | 341315 | | replace with 354626 | | 37 |
| X | 341315 | | | replace with 354626 | 39 |
| Y | 354626:341315 | | | | 34 |
| Z | 116847 | | | | 9 |

A, E, I, M, Q and U - cells were treated at time 0 with one strand; after four hours, the other strand was added.
B, F, J, N, R and V - cells were treated at time 0 with one strand; after 1 hour, the first treatment is removed and replaced with the one containing the other strand.
C, G, K, O, S and W - cells were treated at time 0 with one strand; after 2 hours, the first treatment is removed and replaced with the one containing the other strand.
D, H, L, P, T and X - cells were treated at time 0 with one strand; after 4 hours, the first treatment is removed and replaced with the one containing the other strand.
Y and Z - these were treated at time 0 and left untouched until lysis time.

Oligomeric siRNA compound combinations 354626:341315 show activity in this free uptake system. Sequentially treating with the sense strand of these duplexes first followed by the antisense strand shows good activity at every time point tested.

Even further, sequentially treating with compound 341315, a sense strand having a phosphorothioate backbone and which is fully modified with OMe at the 2′ positions, followed by either the antisense strand of compound 303912 or 354626 (differeing in the 5′ terminal moitety) show equally potent results, suggesting that the 5′ terminus is not the determinant factor in target reduction for these constructs.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method of identifying oligomeric compounds having bioactivity in vivo comprising:
contacting a bioindicative cell with one or more pairs of candidate oligomeric compounds in vitro, wherein the bioindicative cell is contacted with a first oligomeric compound having a sense strand orientation, and the bioindicative cell is contacted with a second oligomeric compound having an antisense strand orientation, wherein the bioindicative cell is contacted with the second oligomeric compound at least one hour after the bioindicative cell is contacted with the first oligomeric compound, and wherein at least a portion of the second oligomeric compound is capable of hybridizing with at least a portion of the first oligomeric compound; and
determining whether the bioindicative cell has an altered phenotype, wherein if the bioindicative cell has an altered phenotype, one or more of the pairs of candidate oligomeric compounds comprises in vivo bioactivity.

2. A method of claim 1 wherein the first and second oligomeric compounds are small interfering RNA.

3. A method of claim 1 wherein the contacting occurs in the absence of a transfection reagent.

4. A method of claim 1 wherein the bioindicative cell is a mammalian tissue-derived cell.

5. A method of claim 4 wherein the mammalian tissue-derived cell is a primary hepatocyte, primary keratinocyte, primary macrophage, primary fibroblast, primary pancreatic cell, or a stem cell.

6. A method of claim 4 wherein the mammalian tissue-derived cell is a rodent primary hepatocyte.

7. A method of claim 6 wherein the rodent is a mouse.

8. A method of claim 6 wherein the rodent is a rat.

9. A method of claim 4 wherein the mammalian tissue-derived cell is a primate primary hepatocyte.

10. A method of claim 9 wherein the primate is a Cynomolgus monkey.

11. A method of claim 9 wherein the primate is a human.

12. A method of claim 1 wherein the altered phenotype is an increase in uptake of the candidate oligomeric compounds, decrease in expression of the mRNA produced from the gene to which the candidate oligomeric compounds are targeted, or decrease in expression of the protein encoded by the gene or mRNA to which the candidate oligomeric compounds are targeted.

13. A method of claim 1 wherein the bioindicative cell is contacted with the second oligomeric compound at least two hours after the bioindicative cell is contacted with the first oligomeric compound.

14. A method of claim 1 wherein the bioindicative cell is contacted with the second oligomeric compound between two hours and four hours after the bioindicative cell is contacted with the first oligomeric compound.

15. A method of claim 1 wherein each of the first and second oligomeric compounds comprises 10 to 40 nucleotides.

16. A method of claim 1 wherein each of the first and second oligomeric compounds comprises 18 to 30 nucleotides.

17. A method of claim 1 wherein each of the first and second oligomeric compounds comprises 21 to 24 nucleotides.

18. A method of claim 1 wherein at least a portion of the second oligomeric compound is complementary to and capable of hybridizing to a selected target nucleic acid, the second oligomeric compound comprises a plurality of linked nucleosides linked by internucleoside linking groups, the first oligomeric compound comprises a plurality of linked nucleosides linked by internucleoside linking groups and wherein essentially each of the nucleosides is other than 2′-OH and have 3′-endo conformational geometry, and the first and second oligomeric compounds optionally comprise a phosphate group, a 3′-overhang, or a conjugate group.

19. A method of claim 18 wherein each of the nucleosides of the second oligomeric compound comprise a B-D-ribofuranose sugar group.

20. A method of claim 18 wherein the 3′-terminus of the second oligomeric compound comprises a stabilizing or conjugate group.

21. A method of claim 20 wherein the stabilizing group is a capping group or a dTdT dimer.

22. A method of claim 20 wherein the 3′-terminus of the second oligomeric compound comprises a conjugate group.

23. A method of claim 18 wherein the second oligomeric compound comprises a 5′-phosphate group.

24. A method of claim 18 wherein the 5′-terminus of the second oligomeric compound comprises a stabilizing or conjugate group.

25. A method of claim 24 wherein the stabilizing group is a capping group.

26. A method of claim 24 wherein the 5′-terminus of the second oligomeric compound comprises a conjugate group.

27. A method of claim 18 wherein the first oligomeric compound comprises a 5′-phosphate group.

28. A method of claim 18 wherein each of the internucleoside linking groups of the second oligomeric compound is, independently, a phosphodiester or a phosphorothioate.

29. A method of claim 28 wherein each of the internucleoside linking groups of the second oligomeric compound is a phosphodiester.

30. A method of claim 28 wherein each of the internucleoside linking groups of the second oligomeric compound is a phosphorothioate.

31. A method of claim 18 wherein each of the internucleoside linking groups of the first oligomeric compound is, independently, a phosphodiester or a phosphorothioate.

32. A method of claim 31 wherein each of the internucleoside linking groups of the first oligomeric compound is a phosphodiester.

33. A method of claim 31 wherein each of the internucleoside linking groups of the first oligomeric compound is a phosphorothioate.

34. A method of claim 18 wherein the 3′-terminus of the first oligomeric compound comprises a stabilizing or conjugate group.

35. A method of claim 34 wherein the stabilizing group is a capping group or a dTdT dimer.

36. A method of claim 34 wherein the 3′-terminus of the first oligomeric compound comprises a conjugate group.

37. A method of claim 18 wherein the 5′-terminus of the first oligomeric compound comprises a stabilizing or conjugate group.

38. A method of claim 37 wherein the stabilizing group is a capping group.

39. A method of claim 37 wherein the 5′-terminus of the first oligomeric compound comprises a conjugate group.

40. A method of claim 18 wherein each of the nucleosides of the first oligomeric compound is a nucleoside having 3′-endo conformational geometry.

41. A method of claim 18 wherein each of the nucleosides having 3′-endo conformnational geometry comprises a 2′-substitutuent group.

42. A method of claim 41 wherein each of the 2′-substituent groups is, independently, —F, —O—CH₂CH₂—O—CH₃, —O—CH₃, —O—CH₂—CH═CH₂ or a group having one of formula Iₐ or IIₐ: 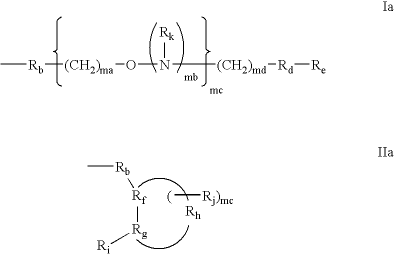
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ; 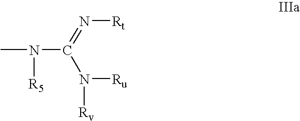
Rₚ and R_{q} are each independently hydrogen or C₁-C₁₀ alkyl;
Rᵣ is —Rₓ—R_{y};
each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent group is hydroxyl, amino, alkoxy, carboxy, benzyl, phenyl, nitro, thiol, thioalkoxy, halogen, alkyl, aryl, alkenyl, or alkynyl;
or optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₚ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent group is hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino, or acyl where the acyl is an acid amide or an ester;
or Rₘ and Rₙ, together, are a nitrogen protecting group, are joined in a ring structure that optionally includes an additional heteroatom selected from N and O or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein the heteroatoms are oxygen, nitrogen, or sulfur and wherein the ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;
mₐ is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

43. A method of claim 41 wherein each of the 2′-substituent groups is, independently, —F, —O—CH₂CH₂—O—CH₃, —O—CH₃, —O—CH₂—CH═CH₂ or —O—CH₂—CH—CH₂—NH(Rⱼ) where Rⱼ is H or C₁-C₁₀ alkyl.

44. A method of claim 41 wherein each of the 2′-substituent groups is, independently, —F, —O—CH₂CH₂—O—CH₃ or —O—CH₃.

45. A method of claim 44 wherein each of the 2′-substituent groups is —O—CH₃.

46. A method of claim 45 wherein each of the internucleoside linking groups of the second oligomeric compound is a phosphodiester.

47. A method of claim 46 wherein each of the internucleoside linking groups of the first oligomeric compound is a phosphodiester.

48. A method of claim 46 wherein each of the internucleoside linking groups of the first oligomeric compound is a phosphorothioate.

49. A method of claim 45 wherein each of the internucleoside linking groups of the second oligomeric compound is a phosphorothioate.

50. A method of claim 49 wherein each of the internucleoside linking groups of the first oligomeric compound is a phosphodiester.

51. A method of claim 49 wherein each of the internucleoside linking groups of the first oligomeric compound is a phosphorothioate.

52. A method of claim 18 wherein the first and second oligomeric compounds have 3′-dTdT overhangs.

53. A method of claim 18 wherein the first and second oligomeric compounds have blunt ends.

54. A method of claim 1 wherein at least one oligomeric compound comprises at least one terminal cap moiety.

55. A method of claim 54 wherein the terminal cap moiety is attached to one or both of the 3′-terminal and 5′-terminal ends of the at least one oligomeric compound.

56. A method of claim 55 wherein the terminal cap moiety is an inverted deoxy abasic moiety.

57. A kit comprising an assay platform, a bioindicative cell, and one or more bioactive pairs of oligomeric compounds which comprise a first oligomeric compound having a sense strand orientation and a second oligomeric compound having an antisense strand orientation, wherein at least a portion of the second oligomeric compound is capable of hybridizing with at least a portion of the first oligomeric compound.


SEQUENCE LISTING (47 sequences)

SEQ ID NO: 1 - DNA, 24 bp, Artificial Sequence
  Description of Artificial Sequence PTEN forward primer
atgacaatcatgttgcagcaattc

SEQ ID NO: 2 - DNA, 25 bp, Artificial Sequence
  Description of Artificial Sequence PTEN reverse primer
cgatgcaataaatatgcacaaatca

SEQ ID NO: 3 - DNA, 26 bp, Artificial Sequence
  Description of Artificial Sequence PTEN probe
gtaaagctggaaagggacggactggt

SEQ ID NO: 4 - DNA, 21 bp, Artificial Sequence
  Description of Artificial Sequence fas forward primer
tccaagacacagctgagcaga

SEQ ID NO: 5 - DNA, 22 bp, Artificial Sequence
  Description of Artificial Sequence fas reverse primer
tgcatcactcttcccatgagat

SEQ ID NO: 6 - DNA, 26 bp, Artificial Sequence
  Description of Artificial Sequence fas probe
agtccagctgctcctgtgctggtacc

SEQ ID NO: 7 - DNA, 19 bp, Artificial Sequence
  Description of Artificial Sequence apolipoprotein b forward primer
cgtgggctccagcattcta

SEQ ID NO: 8 - DNA, 21 bp, Artificial Sequence
  Description of Artificial Sequence apolipoprotein b reverse primer
agtcatttctgcctttgcgtc

SEQ ID NO: 9 - DNA, 22 bp, Artificial Sequence
  Description of Artificial Sequence apolipoprotein b probe
ccaatggtcgggcactgctcaa

SEQ ID NO: 10 - DNA, 21 bp, Artificial Sequence
  Description of Artificial Sequence microsomal triglyceride transfer protein forward primer
gagcggtctggatttacaacg

SEQ ID NO: 11 - DNA, 24 bp, Artificial Sequence
  Description of Artificial Sequence microsomal triglyceride transfer protein reverse primer
aggtagtgacagatgtggcttttg

SEQ ID NO: 12 - DNA, 23 bp, Artificial Sequence
  Description of Artificial Sequence microsomal triglyceride transfer protein probe
caaaccaggtgctgggcgtcagt

SEQ ID NO: 13 - DNA, 15 bp, Artificial Sequence
  Description of Artificial Sequence murine cyclophilin a forward primer
tcgccgcttgctgca

SEQ ID NO: 14 - DNA, 17 bp, Artificial Sequence
  Description of Artificial Sequence murine cyclophilin a reverse primer
atcggccgtgatgtcga

SEQ ID NO: 15 - DNA, 23 bp, Artificial Sequence
  Description of Artificial Sequence murine cyclophilin a probe
ccatggtcaaccccaccgtgttc

SEQ ID NO: 16 - DNA, 20 bp, Artificial Sequence
  Description of Artificial Sequence PTEN antisense
ctgctagcctctggatttga

SEQ ID NO: 17 - DNA, 20 bp, Artificial Sequence
  Description of Artificial Sequence PTEN antisense
cttctggcatccggtttaga

SEQ ID NO: 18 - DNA, 20 bp, Artificial Sequence
  Description of Artificial Sequence PTEN antisense
ctgctagcctctggatttga

SEQ ID NO: 19 - DNA, 20 bp, Artificial Sequence
  Description of Artificial Sequence PTEN antisense
cttctggcatccggtttaga

SEQ ID NO: 20 - DNA, 20 bp, Artificial Sequence
  Description of Artificial Sequence Fas antisense
tccagcactttcttttccgg

SEQ ID NO: 21 - DNA, 20 bp, Artificial Sequence
  Description of Artificial Sequence Fas antisense
tccatctccttttatgccgg

SEQ ID NO: 22 - DNA, 20 bp, Artificial Sequence
  Description of Artificial Sequence MTTP antisense
cccagcacctggtttgccgt

SEQ ID NO: 23 - DNA, 20 bp, Artificial Sequence
  Description of Artificial Sequence Apo B antisense
gtccctgaagatgtcaatgc

SEQ ID NO: 24 - DNA, 21 bp, Artificial Sequence
  Description of Artificial Sequence PTEN antisense
cugcuagccucuggauuugtt

SEQ ID NO: 25 - DNA, 21 bp, Artificial Sequence
  Description of Artificial Sequence PTEN sense
caaauccagaggcuagcagtt

SEQ ID NO: 26 - DNA, 21 bp, Artificial Sequence
  Description of Artificial Sequence PTEN antisense
cuucuggcauccgguuuagtt

SEQ ID NO: 27 - DNA, 21 bp, Artificial Sequence
  Description of Artificial Sequence PTEN sense
cuaaaccggaugccagaagtt

SEQ ID NO: 28 - RNA, 21 nt, Artificial Sequence
  Description of Artificial Sequence PTEN antisense
cugcuagccucuggauuugac

SEQ ID NO: 29 - RNA, 21 nt, Artificial Sequence
  Description of Artificial Sequence PTEN sense
gucaaauccagaggcuagcag

SEQ ID NO: 30 - DNA, 21 bp, Artificial Sequence
  Description of Artificial Sequence Fas antisense
gucugguuugcacuugcactt

SEQ ID NO: 31 - DNA, 21 bp, Artificial Sequence
  Description of Artificial Sequence Fas sense
gugcaagugcaaaccagactt

SEQ ID NO: 32 - DNA, 21 bp, Artificial Sequence
  Description of Artificial Sequence Fas antisense
gugucguguucaguuccactt

SEQ ID NO: 33 - DNA, 21 bp, Artificial Sequence
  Description of Artificial Sequence Fas sense
guggaacugaacacgacactt

SEQ ID NO: 34 - DNA, 20 bp, Artificial Sequence
  Description of Artificial Sequence PTEN antisense
tttgtctctggtccttactt

SEQ ID NO: 35 - DNA, 20 bp, Artificial Sequence
  Description of Artificial Sequence PTEN antisense
tttgtctctggtccttactt

SEQ ID NO: 36 - DNA, 21 bp, Artificial Sequence
  Description of Artificial Sequence PTEN antisense
tttgtctctggtccttacttt

SEQ ID NO: 37 - DNA, 20 bp, Artificial Sequence
  Description of Artificial Sequence PTEN sense
aagtaaggaccagagacaaa

SEQ ID NO: 38 - DNA, 20 bp, Artificial Sequence
  Description of Artificial Sequence PTEN sense
aagtaaggaccagagacaaa

SEQ ID NO: 39 - DNA, 20 bp, Artificial Sequence
  Description of Artificial Sequence PTEN antisense
tttgtctctggtccttactt

SEQ ID NO: 40 - DNA, 20 bp, Artificial Sequence
  Description of Artificial Sequence PTEN sense
aagtaaggaccagagacaaa

SEQ ID NO: 41 - DNA, 20 bp, Artificial Sequence
  Description of Artificial Sequence PTEN sense
aagtaaggaccagagacaaa

SEQ ID NO: 42 - DNA, 21 bp, Artificial Sequence
  Description of Artificial Sequence PTEN antisense
tttgtctctggtccttacttt

SEQ ID NO: 43 - DNA, 20 bp, Artificial Sequence
  Description of Artificial Sequence PTEN antisense
tttgtctctggtccttactt

SEQ ID NO: 44 - DNA, 20 bp, Artificial Sequence
  Description of Artificial Sequence PTEN antisense
tttgtctctggtccttactt

SEQ ID NO: 45 - DNA, 20 bp, Artificial Sequence
  Description of Artificial Sequence PTEN sense
aagcaacgagaagcgataaa

SEQ ID NO: 46 - RNA, 12 nt, Artificial Sequence
  Description of Artificial Sequence dodecamer oligonucleotide
cgcgaauucgcg

SEQ ID NO: 47 - RNA, 12 nt, Artificial Sequence
  Description of Artificial Sequence dodecamer oligonucleotide
gcgcuuaagcgc